SEQUENCE LISTING (5 sequences)

SEQ ID NO: 1 - protein, 604 aa, synthetic construct
MGSSHHHHHHSSGLVPRGSHMDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASNGGGKQALETVQRLLPVLCQDHGGGSGGMVSKGEELFTGVVPILVELDGDVNGHKFSVSGEGEGDATYGKLTLKFICTTGKLPVPWPTLVTTFAYGLQCFARYPDHMKQHDFFKSAMPEGYVQERTISFKDDGNYKTRAEVKFEGDTLVNRIELKGTDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 2 - protein, 162 aa, synthetic construct
MGSSHHHHHHSSGLVPRGSHMMSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKTWTGQGRTPAVIKKAMDEQGKSLDDFLIKQGSGC

SEQ ID NO: 3 - protein, 395 aa, synthetic construct
MGSSHHHHHHSSGLVPRGSHMMSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKWTGQGRTPAVIKKAMDEQGKSLDDFLIKQEFMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGSGC

SEQ ID NO: 4 - protein, 334 aa, synthetic construct
MGSSHHHHHHSSGLVPRGSHMMEQRITLKDYAMRFGQTKTAKDLGVYQSAINKAIHAGRKIFLTINADGSVYAEEVKPFPSNKKTTAGGSGGPGMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGSGC

SEQ ID NO: 5 - protein, 280 aa, synthetic construct
MGSSHHHHHHSSGLVPRGSHMMLPGCMLMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGGSGGKKPRGRPRKPT

(19) United States
(12) Patent Application Publication
(10) Pub. No.: US 2025/0177947 A1
(43) Pub. Date: Jun. 5, 2025

(54) DNA ANALYSIS KIT FOR ANALYZING DNA THROUGH SCANNING ELECTRON MICROSCOPY, DNA-PROTEIN-POLYMER COMPLEX, COMPOSITION INCLUDING THE DNA- PROTEIN-POLYMER COMPLEX, AND DNA ANALYSIS METHOD USING THE DNA-PROTEIN- POLYMER COMPLEX

(71) Applicants: Sogang University Research Foundation, Seoul (KR); Research & Business Foundation Sungkyunkwan University, Suwon-si (KR)

(72) Inventors: Kyubong JO, Seoul (KR); Jung Heon Lee, Seoul (KR); Chanyoung Noh, Seoul (KR); Priyannth Ramasami Sundharbaabu, Gyeonggi-do (KR); Junhyuck Chang, Gyeonggi-do (KR)

(73) Assignees: Sogang University Research Foundation, Seoul (KR); Research & Business Foundation Sungkyunkwan University, Suwon-si (KR)

(21) Appl. No.: 18/583,491

(22) Filed: Feb. 21, 2024

(30) Foreign Application Priority Data
Dec. 1, 2023 (KR) 10-2023-0172725

(51) Int. Cl. B01J 19/08

(52) U.S. Cl. CPC B01J 19/085; B01J 2219/00576; B01J 2219/00612; B01J 2219/00637

(57) ABSTRACT

Provided are a DNA analysis kit for analyzing DNA through scanning electron microscopy (SEM), a DNA-protein-polymer complex, a composition including the DNA-protein-polymer complex, and a DNA analysis method using the DNA-protein-polymer complex. The DNA-protein-polymer complex includes: a DNA molecule; a DNA-binding protein including a peptide, the peptide having an amino acid sequence capable of binding to the DNA molecule and having at least one functional group in at least one of both terminuses thereof; and a polymer, an anhydride thereof or a salt thereof, capable of binding to the DNA-binding protein by intermolecular electrostatic attraction. The DNA-protein-polymer complex is metal-free and enables high-resolution observation and analysis of DNA molecules having various shapes and lengths at almost real-time speed through SEM.

### CROSS-REFERENCE TO RELATED APPLICATION

This application is based on and claims priority under 35 U.S.C. § 119 to Korean Patent Application No. 10-2023-0172725, filed on Dec. 1, 2023, in the Korean Intellectual Property Office, the disclosure of which is incorporated by reference herein in its entirety.

### INCORPORATION BY REFERENCE OF SEQUENCE LISTING

The content of the electronically submitted sequence listing, file name: PX074791US.xml.xml; size: 6.8 kb; and date of creation: Feb. 5, 2024, filed herewith, is incorporated herein by reference in its entirety.

### BACKGROUND

### 1. Field

The disclosure relates to a DNA analysis kit for analyzing DNA through scanning electron microscopy (SEM), a DNA-protein-polymer complex, a composition including the DNA-protein-polymer complex, and a DNA analysis method using the DNA-protein-polymer complex.

### 2. Description of the Related Art

DNA imaging occupies a pivotal position in genomics. In the past, transmission electron microscopy (TEM) was responsible for visualizing single DNA molecules. However, when TEM is used, DNA imaging is performed by using heavy metals alone or in combination with a shadow casting method. To this end, much time and complicated processes are required to observe DNA molecules through TEM.

Instead of such TEM, fluorescence microscopy (FM), atomic force microscopy (AFM), and the like have been used. Among these, FM-based DNA imaging has been widely adopted due to simplicity, ease of access, and live imaging capability. In addition, FM-based DNA imaging is compatible with microfluidic devices and chemically functionalized surfaces. However, FM has a low resolution, thus making it difficult to observe images of DNA having various shapes and lengths almost as it is.

Alternatively, scanning electron microscopy (SEM) is used. DNA observation through SEM is not only advantageous in terms of high resolution at a nanometer-scaled thickness, but can also be used in substrates such as silicon wafers having various chemically functionalized surfaces instead of carbon films and in microfluidic devices that may be used in the alignment of DNA molecules. However, imaging of DNA molecules having a thickness of 2 nm falls short of detection limitations of existing SEM imaging techniques. To improve the visibility of DNA molecules, methods such as nanoparticle attachment and nanowire growth have been used. However, these methods are mainly used to visualize artificially constructed DNA nanostructures, and have great difficulties when applied to DNA.

Therefore, there is still a need for a novel DNA analysis kit for analyzing DNA through SEM, in which DNA molecules having various shapes and lengths can be observed and analyzed with high resolution at real-time speed through SEM, a DNA complex, a composition including the DNA complex, and a DNA analysis method using the DNA complex.

### SUMMARY

Provided is a DNA analysis kit for analyzing DNA through scanning electron microscopy (SEM).

Provided is a DNA-protein-polymer complex which is metal-free and enables high-resolution observation and analysis of DNA molecules having various shapes and lengths at almost real-time speed through scanning electron microscopy (SEM).

Provided is a composition including the DNA-protein-polymer complex.

Provided is a DNA analysis method using the DNA-protein-polymer complex.

Additional aspects will be set forth in part in the description which follows and, in part, will be apparent from the description, or may be learned by practice of the presented embodiments of the disclosure.

According to an aspect of the disclosure, a DNA analysis kit for analyzing DNA through scanning electron microscopy (SEM) includes
a DNA-binding protein capable of binding to a DNA molecule through at least one of electrostatic interaction, intercalation, and groove binding, and
a polymer, an anhydride thereof or a salt thereof, capable of binding to the DNA-binding protein by intermolecular electrostatic attraction.

The DNA-binding protein may include a fluorescent protein.

The polymer, the anhydride thereof, or the salt thereof may include at least one of structural units represented by Formulae 1 to 9 and have average molecular weight of about 10 Kilodaltons to about 100 Kilodaltons:

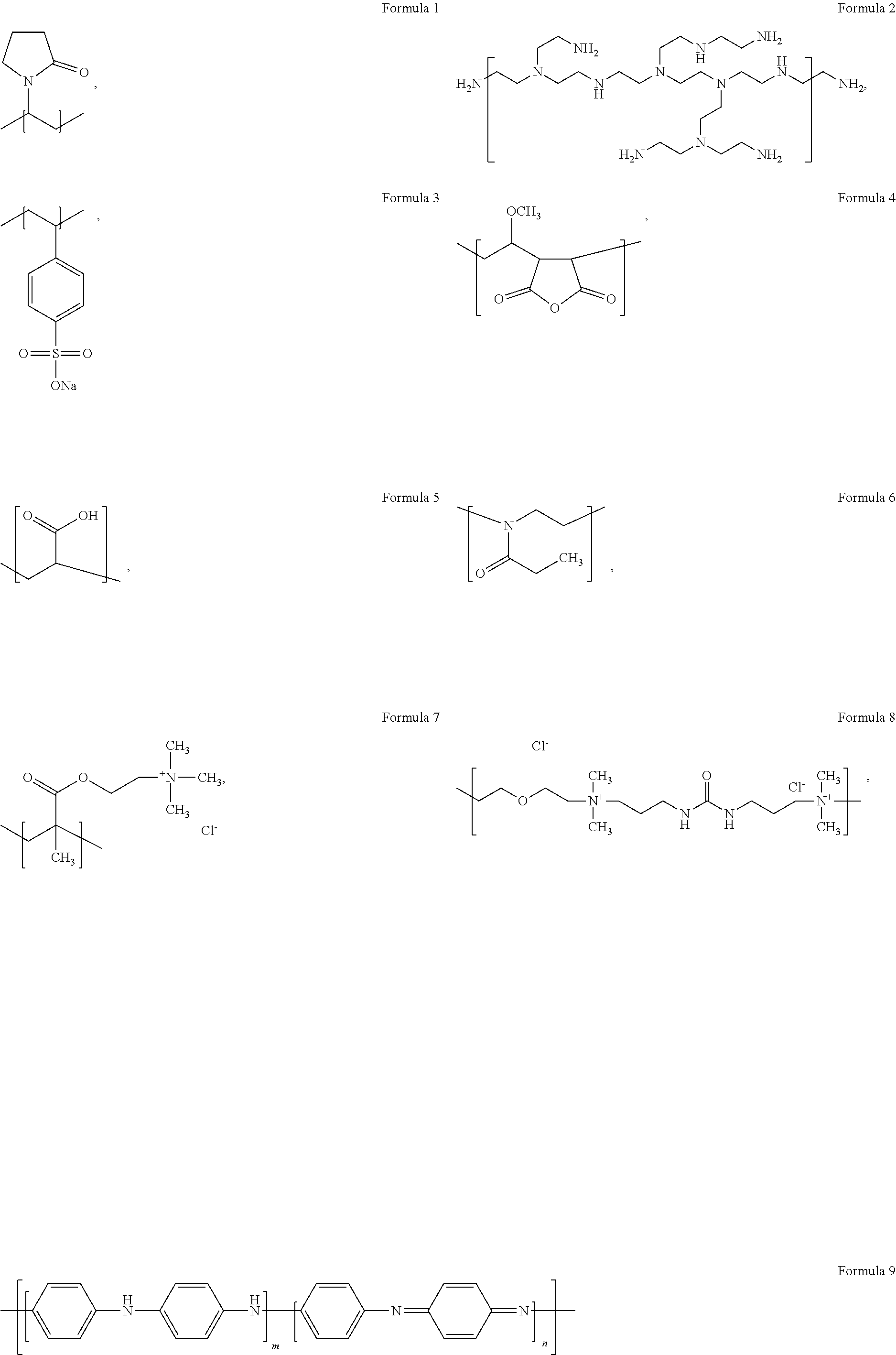

wherein, in Formulae 1 to 9,
m+n may be 1.

According to another aspect of the disclosure, a DNA-protein-polymer complex includes
a DNA molecule,
a DNA-binding protein including a peptide, the peptide having an amino acid sequence capable of binding to the DNA molecule and having at least one functional group in at least one of both terminuses thereof, and
a polymer, an anhydride thereof or a salt thereof, capable of binding to the DNA-binding protein by intermolecular electrostatic attraction.

The polymer may include at least one of an oxygen atom and a nitrogen atom.

The oxygen atom or the nitrogen atom of the polymer is capable of binding to a hydrogen atom of an O—H bond or a hydrogen atom of an N—H bond, contained in the DNA-binding protein, by hydrogen bonding.

The polymer, the anhydride thereof, or the salt thereof may be in the form of aggregates.

The polymer, the anhydride thereof, or the salt thereof may include at least one of structural units represented by Formulae 1 to 9 and have average molecular weight of about 10 Kilodaltons to about 100 Kilodaltons:

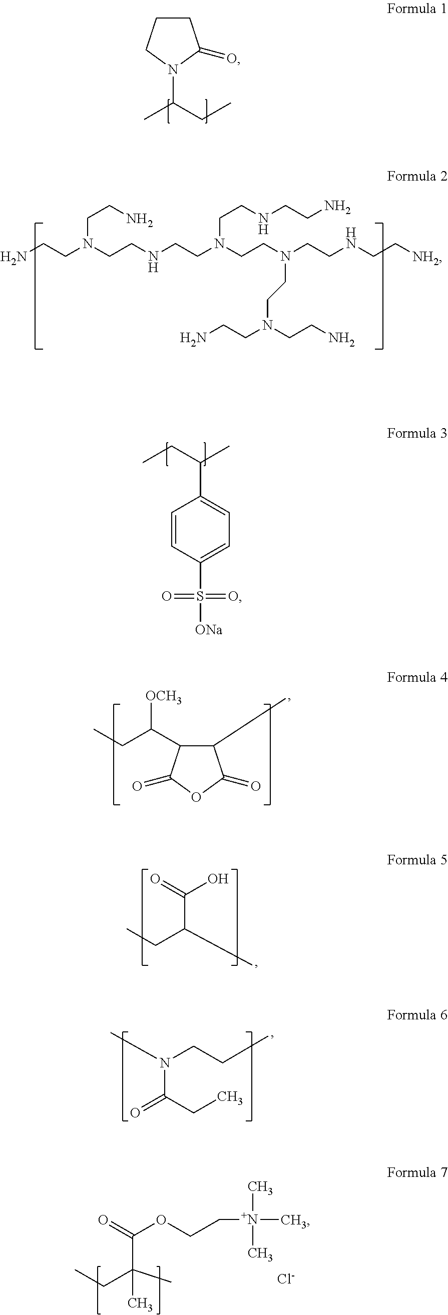

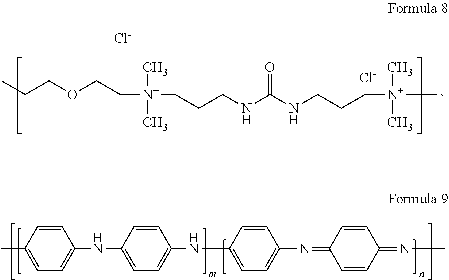

wherein, in Formulae 1 to 9,
m+n may be 1.

The DNA molecule may have a linear, circular, helical, supercoil, Christmas-tree, double-stranded or single-stranded form.

The DNA-binding protein may further include a fluorescent protein.

The DNA-binding protein may be capable of binding to the DNA molecule through at least one of electrostatic interaction, intercalation, and groove binding.

The complex may have a thickness of about 9 nm to about 20 nm.

The complex may be used to stain DNA having a two-dimensional or three-dimensional form and allows the DNA to be visualized through scanning electron microscopy (SEM).

The complex may be used to enable visualization of DNA in a continuous form through SEM.

The complex may be metal-free.

According to another aspect of the disclosure, a composition includes
the above-described DNA-protein-polymer complex and
at least one selected from a solvent, an acid, a base, and a buffer solution.

According to another aspect of the disclosure, a DNA analysis method using the DNA-protein-polymer complex includes
providing a silicon substrate modified to have a positively charged surface, and
applying the above-described composition onto a surface of the silicon substrate and observing a DNA molecule through scanning electron microscopy.

The observing of the DNA molecule through scanning electron microscopy may include:
immobilizing a DNA molecule on and stretching the DNA molecule from the surface of the silicon substrate by using a PDMS microchannel into which the composition is injected, and
removing the PDMS microchannel from the silicon substrate and observing the immobilized and stretched DNA molecule through scanning electron microscopy.

The providing of the silicon substrate modified to have a positively charged surface may include
preparing a silicon substrate having a silicon oxide film formed thereon, and
contacting the silicon substrate having a silicon oxide film formed thereon with an ammonium salt precursor to provide a silicon substrate modified to have a positively charged surface.

The ammonium salt precursor may include at least one selected from ammonia water, an ammonium chloride-based compound, an ammonium sulfate-based compound, an ammonium carbonate-based compound, an ammonium bicarbonate-based compound, and an ammonium acetate-based compound.

The method may further include, before the removal of the PDMS microchannel from the silicon substrate, drying the composition.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a view of a DNA-protein-polymer complex according to an embodiment;

FIG. 2 is a view of a PDMS microchannel device according to an embodiment;

FIG. 3A illustrates the results of observing, through a field emission-scanning electron microscope (FE-SEM), DNA molecules stained using a DNA-mNeonGreen-HMG-PVP complex composition according to Example 7 on a silicon wafer surface-modified with cations according to Reference Example 1;

FIG. 3B illustrates the results of observing, through an FE-SEM, DNA molecules stained using the DNA-mNeonGreen-HMG-PVP complex composition according to Example 7 on a silicon oxide wafer according to Reference Example 2;

FIG. 3C illustrates the results of observing, through an FE-SEM, DNA molecules stained using the DNA-mNeonGreen-HMG-PVP complex composition according to Example 7 on a silicon wafer according to Reference Example 3;

FIG. 3D illustrates the results of observing, through an FE-SEM, DNA molecules stained using the DNA-mNeonGreen-HMG-PVP complex composition according to Example 7 on a Pt-coated silicon wafer according to Reference Example 4;

FIGS. 4A and 4B illustrate the results of observing DNA molecules stained using the DNA-mNeonGreen-HMG-PVP complex composition according to Example 7, through an FE-SEM and a fluorescence microscope (FM), respectively, without using a PDMS microchannel device;

FIG. 5 illustrates the results of observing, through an FE-SEM, DNA molecules stained using the DNA-mNeonGreen-HMG-PVP complex composition according to Example 7, in which a PDMS microchannel device was used;

FIG. 6A illustrates the results of observing, through an FE-SEM, DNA molecules stained using a DNA-mNeonGreen-HMG-PEOX complex composition according to Example 5, in which a PDMS microchannel device was used;

FIG. 6B illustrates the results of observing, through an FE-SEM, DNA molecules stained using the DNA-mNeonGreen-HMG-PEOX complex composition according to Example 5, without using a PDMS microchannel device;

FIGS. 7A and 7B illustrate the results of observing DNA molecules stained using a DNA-protein-polymer complex composition according to Example 1, through an FE-SEM and an FM, respectively;

FIG. 8 illustrates the results of observing DNA molecules stained using a DNA-protein-polymer complex composition according to Example 2, through an FE-SEM;

FIGS. 9A and 9B illustrate the results of observing DNA molecules stained using a DNA-protein-polymer complex composition according to Example 3, through an FE-SEM and an FM, respectively;

FIGS. 10A and 10B illustrate the results of observing DNA molecules stained using a DNA-protein-polymer complex composition according to Example 4, through an FE-SEM and an FM, respectively;

FIG. 11 illustrates the results of observing DNA molecules stained using a DNA-protein-polymer complex composition according to Example 6, through an FE-SEM;

FIG. 12 illustrates the results of observing DNA molecules through an FE-SEM, in which a DNA solution according to Comparative Example 1 was used;

FIGS. 13A and 13B illustrate the results of observing DNA molecules through an FE-SEM and an FM, respectively, in which a DNA-mNeonGreen-HMG mixture according to Comparative Example 3 was used;

FIG. 14 illustrates the results of observing stained DNA molecules through an FE-SEM, in which a DNA-PVP mixture according to Comparative Example 3 was used;

FIG. 15 illustrates the results of observing stained DNA molecules through an FE-SEM, in which a DNA-PANI mixture according to Comparative Example 4 was used;

FIGS. 16A and 16B illustrate the results of observing stained DNA molecules through an FE-SEM and an FM, respectively, in which a DNA-mNeonGreen-HMG-PVP complex composition according to Example 7 was used;

FIG. 16C illustrates the results of observing a DNA-mNeonGreen-HMG-PVP complex through an inverted FE-SEM, in which the DNA-mNeonGreen-HMG-PVP complex composition according to Example 7 was used;

FIG. 16D is a graph using a full width at half maximum macro profile for the DNA-mNeonGreen-HMG-PVP complex according to Example 7; and

FIGS. 17A and 17B illustrate the results of observing DNA molecules stained using the DNA-mNeonGreen-HMG-PVP complex composition, through an FE-SEM and an FM, respectively.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, a DNA analysis kit for analyzing DNA through scanning electron microscopy (SEM) according to an embodiment, a DNA-protein-polymer complex, a composition including the DNA-protein-polymer complex, and a DNA analysis method using the DNA-protein-polymer complex will be described in detail with reference to the accompanying drawings. The following description is provided for illustrative purposes and is not intended to limit the disclosure, and the disclosure is defined only by the scope of the appended claims.

Hereinafter, the case of being “above” or “on” may include not only a case of being directly on while in contact, but also a case of being on while not in contact. An expression in the singular includes an expression in the plural unless the content clearly indicates otherwise. In addition, when a part is referred to as “including” a certain element, it means a case where the part may further include other elements, rather than precluding other elements, unless otherwise specifically stated herein. The term “combination” as used herein includes a mixture, an alloy, a reaction product, or the like unless otherwise specifically stated herein. Terms such as “first,” “second,” “third,” and “fourth” may be used to describe various elements, but such elements must not be limited by these terms. The above terms are used only to distinguish one component from another. The term “or” means “and/or” unless otherwise specified. The term “connected” as used herein may refer to direct connection, indirect connection, or indirect communication. The expressions “an embodiment,” “embodiments,” and the like throughout the present specification mean that a stated specific element may be included in at least one embodiment set forth herein, and may be present or not be present in other embodiments. In addition, it should be understood that elements described herein may be combined in any suitable manner in various embodiments. Unless otherwise defined, technical and scientific terms used herein have the same meaning as commonly understood by one of ordinary skill in the art to which the present application pertains. All cited patents, patent applications, and other references are incorporated herein by reference in their entirety. However, in the event of any conflict or inconsistency between terms used herein and terms of the cited references, the terms used in this specification take precedence over the terms of the cited references. While specific embodiments and implementations have been described, alternatives, modifications, variations, improvements, and substantial equivalents that are currently unpredicted or unpredictable can be made by the applicant or those of ordinary skill in the art. Therefore, the appended claims and claims to be amended are intended to encompass all such alternatives, modifications, variations, improvements, and substantial equivalents.

A DNA analysis kit for analyzing DNA through scanning electron microscopy (SEM), according to an embodiment, may include: a DNA-binding protein capable of binding to a DNA molecule through at least one of electrostatic interaction, intercalation, and groove binding; and a polymer, an anhydride thereof or a salt thereof, capable of binding to the DNA-binding protein by intermolecular electrostatic attraction.

The DNA-binding protein may include a fluorescent protein.

The polymer, the anhydride thereof, or the salt thereof may include at least one of structural units represented by Formulae 1 to 9 and have average molecular weight of about 10 Kilodaltons to about 100 Kilodaltons:

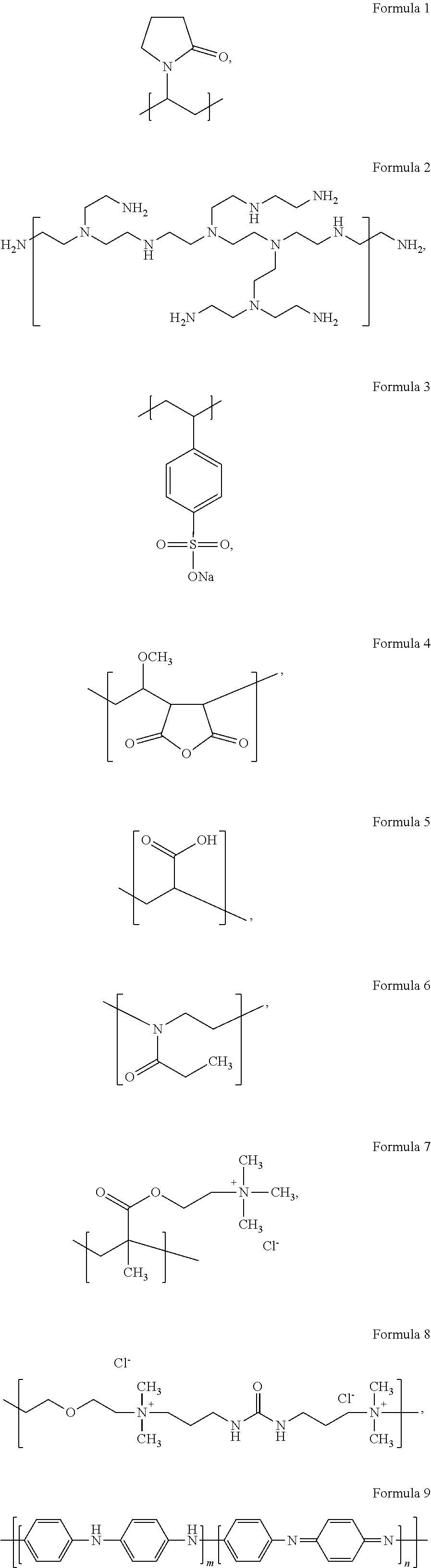

wherein, in Formulae 1 to 9,
m+n may be 1.

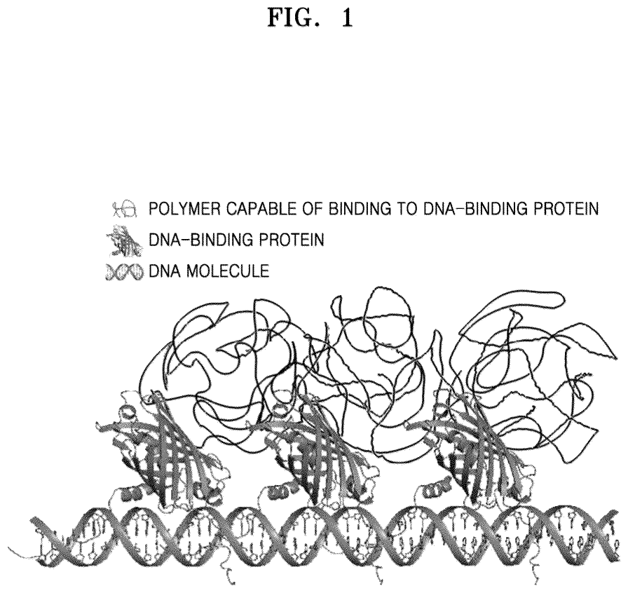

FIG. 1 is a view of a DNA-protein-polymer complex according to an embodiment.

Referring to FIG. 1, the DNA-protein-polymer complex according to an embodiment includes a DNA molecule, a DNA-binding protein capable of binding to the DNA molecule, and a polymer capable of binding to the DNA-binding protein. A complex refers to a structure in which at least two components are combined. The DNA-protein-polymer complex as used herein refers to a structure in which DNA, a protein, and a polymer are combined.

A DNA molecule is a polymer of nucleotides and has a structure with a phosphate bonded to one side of centered deoxyribose and one of four types of nucleobases bonded to the other side thereof. DNA molecules make up the body of genes.

In an embodiment, a DNA molecule may be any DNA molecule regardless of the structure, length and shape thereof. A DNA molecule may be a DNA molecule having a length of several kbps to several Mbps. In an example embodiment, a DNA molecule may be DNA having a nano-sized thickness and a micron-sized length. Non-limiting examples of DNA molecules may include A phage DNA (48.5 kb) or M13mp18 double-stranded DNA. For example, a DNA molecule may have a linear, circular, helical, supercoil, Christmas-tree, double-stranded or single-stranded form.

A DNA-binding protein is a protein having specific or non-specific affinity for a DNA-binding domain and a DNA molecule. In an embodiment, a DNA-binding protein may include a peptide having an amino acid sequence capable of binding to a DNA molecule and at least one functional group in at least one of both terminuses thereof.

The amino acid sequence may include at least one of lysine (Lys), tryptophan (Trp), arginine (Arg), histidine (Hi), phenylalanine (Phe), alanine (Ala), and tyrosine (Tyr). However, the disclosure is not limited thereto and any amino acid sequence available for DNA-binding proteins may be used.

The DNA-binding protein may be capable of binding to a DNA molecule through at least one of electrostatic interaction, intercalation, and groove binding. In an example embodiment, the peptide may interact electrostatically with negatively charged DNA molecules by including positively charged amino acid residues. The peptide that binds to the DNA molecule by electrostatic interaction may include an amino acid sequence including at least one of arginine, histidine, and lysine. The groove binding refers to binding between an exposed base of the DNA molecule and the peptide including the amino acid sequence, and the peptide may bind to DNA through groove binding. Intercalation refers to a phenomenon in which molecules, atoms and ions are inserted between layers of a layered material, and the peptide including the amino acid sequence may be inserted into the DNA molecule. In this regard, the peptide that binds to the DNA molecule by intercalation may include an amino acid sequence including at least one of phenylalanine, tyrosine, and tryptophan, which are aromatic ring-containing amino acids.

The peptide may groove bind or intercalate with the DNA molecule in addition to electrostatic interaction, or may intercalate and groove bind with the DNA molecule in addition to electrostatic interaction.

The peptide may have at least one functional group in at least one of both terminuses, i.e., the N-terminus and the C-terminus, thereof. The functional group may include at least one of a thiol group, amine, a carboxyl group, a cysteine group, an azide group, an alkyne group, a glutaraldehyde group, and an maleimide group. However, the disclosure is not limited thereto and the functional group may include any functional group available for DNA-binding proteins. For example, the peptide may bind to the DNA molecule to thereby provide the above-described functional group for the DNA molecule.

In an example embodiment, the DNA-binding protein may include histone-like nucleotide structural proteins such as Truncated TALE (tTALE), H—NS, Cro, HMG, and the like.

In an example embodiment, the DNA-binding protein may further include a fluorescent protein. The fluorescent protein is not particularly limited and may include all known fluorescent proteins. The fluorescent protein serves as an excellent docking site for a polymer, which will be described below. In addition, oligomerization of the fluorescent protein may improve binding strength with the polymer as described below and may exist as aggregates around the DNA molecule along with the polymer as described below.

In an example embodiment, the DNA-binding protein may bind to the fluorescent protein via a linker or may directly bind to DBP and FP without a linker. Non-limiting examples of DNA-binding protein-fluorescent protein may include Truncated TALE (tTALE)-eGFP, H—NS-mScarlet, Cro-mNeonGreen, and mNeonGreen-HMG. These DNA binding protein-fluorescent proteins may affect brightness needed to visualize DNA molecules through electron microscopy and stretching of DNA molecules.

In this specification, the polymer capable of binding to the DNA-binding protein may include an anhydride thereof or a salt thereof, in addition to the polymer. The salt may be an inorganic salt or an organic salt. For example, the salt may be a cationic inorganic salt.

In an example embodiment, the polymer may include at least one of an oxygen atom and a nitrogen atom.

In an example embodiment, the oxygen atom or the nitrogen atom of the polymer may be capable of binding to a hydrogen atom in an O—H bond or a hydrogen atom in an N—H bond, contained in the DNA-binding protein, by hydrogen bonding. Such hydrogen bonding corresponds to intermolecular bonding and is distinguished from chemical bonding.

In an example embodiment, the polymer, the anhydride thereof, or the salt thereof may be in the form of aggregates.

In an example embodiment, the polymer, the anhydride thereof, or the salt thereof may include at least one of structural units represented by Formulae 1 to 9:

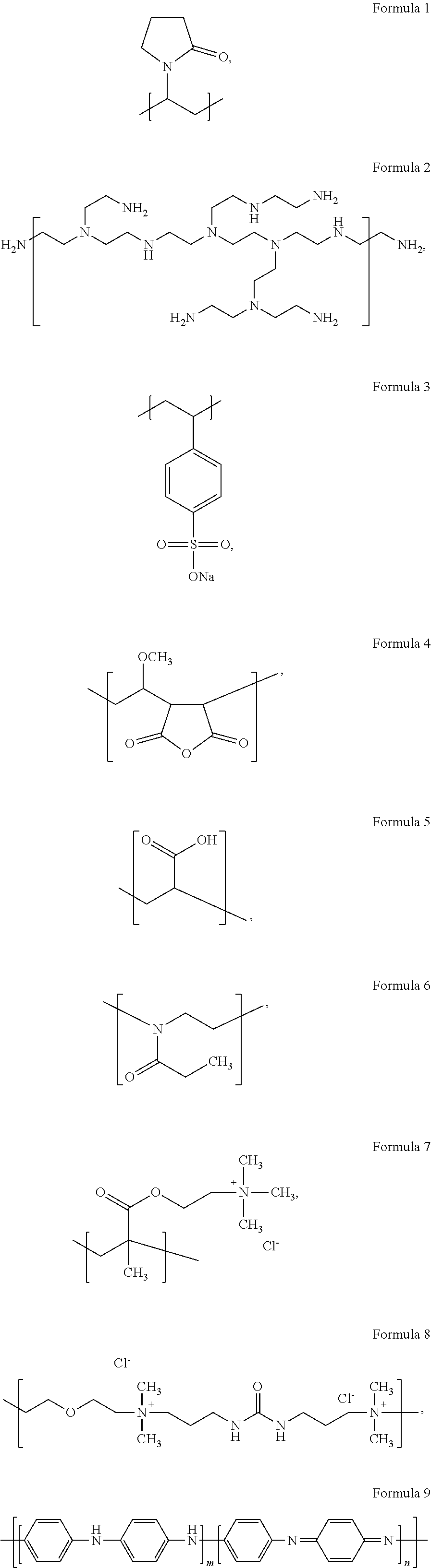

wherein, in Formulae 1 to 9,
m+n may be 1.

In an example embodiment, the polymer, the anhydride thereof, or the salt thereof may have average molecular weight (Mw) of about 10 Kilodaltons to about 100 Kilodaltons. For example, the average molecular weight (Mw) of the polymer, the anhydride thereof, or the salt thereof may be in a range of about 20 Kilodaltons to about 100 Kilodaltons, about 30 Kilodaltons to about 100 Kilodaltons, about 40 Kilodaltons to about 100 Kilodaltons, or about 40 Kilodaltons to about 90 Kilodaltons.

In an example embodiment, the polymer, the anhydride thereof, or the salt thereof may stain the DNA molecule black along with the above-described DNA-binding protein, thereby contributing to visualization of the DNA molecule through SEM.

In an example embodiment, the complex may have a thickness of about 9 nm to about 20 nm. In an example embodiment, the complex may have an average thickness of about 15.0±4.0 nm.

The complex according to an embodiment may be used to stain DNA having a two-dimensional or three-dimensional form and allow the DNA to be visualized through SEM.

The complex according to an embodiment may be used to enable visualization of DNA in a continuous form through SEM.

The complex according to an embodiment may be metal-free. Therefore, much time and complicated processes are not required to observe DNA molecules through transmission electron microscopy (TEM).

A composition according to another embodiment may include: the above-described DNA-protein-polymer complex; and at least one selected from a solvent, an acid, a base, and a buffer solution. The composition may be prepared by adding the above-described DNA-protein-polymer complex to a solvent, a buffer solution, or a mixture thereof, and adding an acid and/or a base thereto. In addition, the composition may further include other additives that may be used in the art. The amounts of the solvent, acid, base, and buffer solution included in the composition may be appropriately adjusted depending on required performance. In other embodiments, the composition may be mixed with a sample.

A DNA analysis method using a DNA-protein-polymer complex, according to another embodiment, may include: providing a silicon substrate modified to have a positively charged surface; and applying the above-described composition onto a surface of the silicon substrate and observing a DNA molecule through scanning electron microscopy.

The term “applying” as used herein refers to any surface application method that may be used in the art, such as “adding dropwise.”

The observing of the DNA molecule through scanning electron microscopy may include: immobilizing a DNA molecule on and stretching the DNA molecule from the surface of the silicon substrate by using a PDMS microchannel into which the composition is injected; and removing the PDMS microchannel from the silicon substrate and observing the immobilized and stretched DNA molecule through scanning electron microscopy.

The silicon substrate modified to have a positively charged surface serves to immobilize the DNA molecule. In comparison, a surface of a glass substrate used in fluorescence microscopy (FM) imaging induces electron charging effects, and thus is not suitable for observing DNA molecules through SEM. In addition, when DNA molecules are observed through SEM, a substrate having a surface coated with a conductive metal such as gold (Au) or platinum (Pt) is not suitable since an image of 2 nm-thick DNA molecules is buried and appears blurred. The silicon substrate used in the disclosure has semiconductor properties, and thus, effective charge transfer is possible.

The providing of the silicon substrate modified to have a positively charged surface may include: preparing a silicon substrate having a silicon oxide film formed thereon; and contacting the silicon substrate having a silicon oxide film formed thereon with an ammonium salt precursor to thereby provide a silicon substrate modified to have a positively charged surface.

A silicon substrate having a silicon oxide film formed thereon, of which the surface is modified with cations of an ammonium salt, serves to immobilize the backbone of the DNA molecule. The thickness of the silicon oxide film may be in a range of several nanometers to tens of nanometers. According to the surface modification with a positive charge, the thickness of the silicon oxide film may be appropriately adjusted within a range that maintains the positive charge.

The ammonium salt precursor may include at least one selected from ammonia water, an ammonium chloride-based compound, an ammonium sulfate-based compound, an ammonium carbonate-based compound, an ammonium bicarbonate-based compound, and an ammonium acetate-based compound. For example, the ammonium salt precursor may be an ammonium chloride-based compound. For example, the ammonium salt precursor may be N-trimethoxysilylpropyl-N,N,N-trimethyl ammonium chloride.

In an example embodiment, the DNA analysis method may further include, before the removal of the PDMS microchannel from the silicon substrate, drying the composition.

The PDMS microchannel is a polydimethylsiloxane microchannel and serves to guide the DNA-protein-polymer complex composition therethrough. The PDMS microchannel is configured to stretch the DNA molecule and allows the DNA molecule to appear as a parallel, well-arranged image when observed through SEM. After the DNA-protein-polymer complex composition is dried at room temperature for about 30 minutes to about 1 hour, an image is observed through SEM.

Hereinafter, examples and comparative examples of the disclosure will be described. However, the following examples are provided for illustrative purposes only and are not intended to limit the disclosure.

### Examples

### Materials

Unless otherwise specified, the following materials were used.

A QIAprep spin miniprep kit and an Ni-NTA agarose resin were purchased from Qiagen (Hilden, Germany). λ phage DNA (48.5 kb), Nb. BssSI, M13mp18 double-stranded DNA (7.2 kb), and DNA polymerase I were purchased from New England Biolabs (Ipswich, MA). T4 GT7 DNA was purchased from Nippongene (Tokyo, Japan). Trypsin derived from porcine pancreas was purchased from Sigma-Aldrich (St. Louis, Missouri). Colcemid was purchased from Roche (Basel, Switzerland). The AccuRapid cloning kit and DEPC-DW were purchased from Bioneer (Daejeon, Korea). DMEM (high concentration glucose, pyruvate), FBS (fetal bovine serum, qualified, USA), antibiotic-antimycotic (100×), and trypsin-EDTA (0.25%, phenol red) were purchased from gibco (Waltham, MA). Ultra LMP agarose was purchased from Invitrogen (Waltham, MA). Proteinase K was purchased from Enzynomics (Daejeon, Korea). AMPure XP beads were purchased from Beckman Coulter (Brea, California). Polyvinylpyrrolidone (PVP, weight average molecular weight: 40,000), poly(2-ethyl-2-oxazoline), polyaniline (emeraldine salt), a 25 nm MCE membrane, and a formaldehyde solution (37% in water) were purchased from Merck Millipore (Burlington, MA). Formamide was purchased from JUNSE (Tokyo, Japan). Qdot 585 streptavidin conjugate was purchased from Thermo Fisher Scientific (Waltham, MA). A SU-8 photoresist (2005, 2015) and a SU-8 developer were purchased from Kayaku Advanced Materials (Westborough, MA). PDMS and a curing agent thereof were purchased from K1 solution (Gwangmyeong, Korea). Ethyl alcohol (99.9%), methyl alcohol, sulfuric acid, and hydrogen peroxide were purchased from Jin Chemical (Siheung, Korea). Glacial acetic acid was purchased from duksan reagents (Ansan, Korea). N-trimethoxysilylpropyl-N,N,N-trimethyl ammonium chloride in 50% methanol was purchased from Gelest (Morrisville, PA).

### PDMS Microchannel

A PDMS microchannel was manufactured as described in “Counting DNA molecules on a microchannel surface for quantitative analysis, Talanta 2023, 252:123826.”

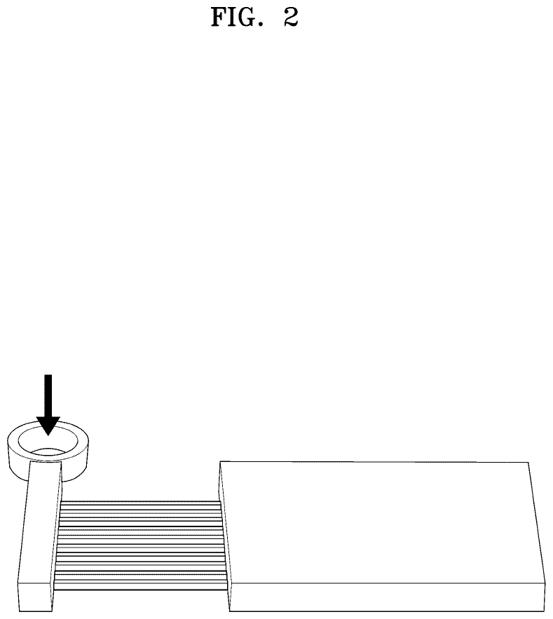

A PDMS device was manufactured by using a standard soft-lithography replica molding technique. First, a microchannel template was deposited as two layers on a silicon wafer according to repeated photolithography procedures, and then followed a protocol described in the Kayaku Advanced Materials SU-8 2000 datasheet. Next, a 20 μm-thick layer of SU-8 2015 photoresist was applied onto the silicon wafer by using a spin coater (Midas System SPIN-1200D, Daejeon, Korea). Subsequently, the spin-coated wafer was exposed to 350 nm radiation by using an aligner (Midas System MDA-400LJ, Daejeon, Korea) through a mask. The wafer was baked and developed by using an SU-8 developer. Then, a maskless lithography system available from SmartPrint (SmartForce Technologies, La Tronche, France) was used to enable fine patterning of the second layer on the developed wafer. SmartPrint is compatible with g-line photoresist, and thus, SU-8 TF 6002 was used. After the template wafer was fabricated, the microchannel template placed on the silicon wafer was coated with a mixture of a PDMS pre-polymer and a curing agent (a weight ratio of 10:1) and incubated at 65° C. for 12 hours. The cured PDMS was separated from the wafer, and then a chamber was created by physically punching a channel into the PDMS. Subsequently, the PDMS microchannel was oxidized at 100 W for 30 seconds by using an air plasma generator (Femto Science Cute Basic, Korea). Finally, the PDMS device was washed and stored in deionized water to fabricate a PDMS microchannel as illustrated in FIG. 2 (100 μm×2.4 μm).

### (DNA-Binding Protein)

### Preparation Example 1: Truncated TALE (tTALE)-emGFP

A tTALE-emGFP plasmid was constructed as described in “Truncated TALE-FP as DNA Staining Dye in a High-salt Buffer. Sci Rep-Uk 9, 17197 (2019)” and “21 Fluorescent Protein-Based DNA Staining Dyes. Molecules 27 (2022).”

The tTALE-emGFP plasmid was produced through an extension polymerase chain reaction for linking an emGFP fluorescent protein to the C-terminus region of DNA-binding protein TALE. A bridging amino acid sequence was GGSGG. The tTALE-emGFP plasmid construct was transformed into an E. coli BL21 (DE3) strain by using standard cloning procedures. The amino acid sequence for tTALE-emGFP is shown in Table 1.

### Preparation Example 2: H—NS

An H—NS plasmid was constructed by amplifying the H—NS gene from H—NS-mCherry as described in Analyst 144, 921-927 (2019). Plasmid pET15b was digested with restriction enzymes Ndel (CA∧TATG) and BamHI (G∧GATCC). Then, the H—NS gene was inserted and ligated into the plasmid by using an AccuRapid cloning kit to produce the H—NS plasmid. The H—NS plasmid construct was transformed into an E. coli BL21 (DE3) strain by using standard cloning procedures. The amino acid sequence for H—NS is shown in Table 1.

### Preparation Example 3: H—NS-mScarlet

An H—NS-mScarlet plasmid was constructed as described in “AT-specific DNA visualization revisits the directionality of bacteriophage lambda DNA ejection. Nucleic Acids Research (2023).”

An H—NS plasmid was digested with BamHI (G∧GATCC). Then, an amplified mScarlet gene was also inserted and ligated into the H—NS plasmid by using an AcuRapid cloning kit. The H—NS plasmid construct was transformed into an E. coli BL21 (DE3) strain by using standard cloning procedures. The amino acid sequence for H—NS-mScarlet is shown in Table 1.

### Preparation Example 4: Cro-mNeonGreen

A Cro-mNeonGreen plasmid was constructed as described in “Negative nanopore sequencing for mapping biochemical processes on DNA molecules. Chemical Communications 59, 9388-9391 (2023).”

Cro, which is a gene encoding a DNA-binding protein, was inserted into a mNeonGreen plasmid by using a cassette vector system to produce a Cro-mNeonGreen plasmid.

The mNeonGreen plasmid was produced by inserting the mNeonGreen gene between Ndel (CA∧TATG) and Xmal (C∧CCGGG) restriction sites in pET15b vector. The Cro gene was amplified by PCR using bacteriophage A DNA as a template. The Cro-mNeonGreen plasmid construct was transformed into an E. coli BL21 (DE3) strain by using standard cloning procedures. The amino acid sequence for Cro-mNeonGreen is shown in Table 1.

### Preparation Example 5: mNeonGreen-HMG

The mNeongreen-HMG plasmid was constructed as described in “21 Fluorescent Protein-Based DNA Staining Dyes. Molecules 2022, 27 (16).”

The mNeongreen-HMG plasmid was produced by using an extension polymerase chain reaction for linking a fluorescent protein to the N-terminus region of high mobility group (HMG), which is a DNA-binding protein. A bridging amino acid sequence was GGSGG. For mNeongreen, Lamp1-mNeonGreen provided by Dorus Gadella (Addgene plasmid #98882) was used. The Addgene construct served as a template to produce an extension PCR product including HMG and GGSGG linker. The amino acid sequence for mNeongreen-HMG is shown in Table 1.

The mNeongreen-HMG plasmid construct was transformed into an E. coli BL21 (DE3) strain by using standard cloning procedures. To express the DNA-binding protein, a frozen BL21 cell stock including the plasmid was quickly thawed, and then cultured overnight at 37° C. in a fresh LB medium supplemented with ampicillin. When the culture reached confluency, 1 mL of the overnight culture was transferred to 100 ml of a fresh LB medium containing ampicillin and incubated at 37° C. until the optical density at 600 nm (OD600) reached 0.4-0.6. Thereafter, the protein was induced by adding 1 mM IPTG and culturing overnight at 20-25° C. and 200 rpm on a shaker (Dlab Scientific Co., Beijing, China). To perform cell lysis, sonication was performed for 15 minutes, and the resulting cell debris was centrifuged at 4° C. and 10,000 rpm for 10 minutes. The DNA-binding protein was purified by affinity chromatography for Ni-NTA agarose resin. A mixture of the cell proteins and the resin was incubated at 4° C. for 3 hours on a shaker platform. Proteins present in a lysate bound to the Ni-NTA agarose resin were loaded onto an affinity column. Then, the column was washed with a protein wash buffer solution (50 mM Na₂HPO₄, 300 mM NaCl, and 20 mM imidazole, pH 8.0), and the DNA-binding protein was eluted with a protein elution buffer solution (50 mM Na₂HPO₄, 300 mM NaCl, and 250 mM imidazole, pH 8.0). The purified protein was buffer-exchanged with 1×PBS (phosphate-buffered saline: 137 mM NaCl, 2.7 mM KCl, 10 mM Na₂HPO₄, and 1.8 mM KH₂PO₄, pH 7.4) and stored at −20° C. with the addition of glycerol. The absorbance of the purified protein was measured by using a NanoDrop spectrophotometer (Eppendorf, Hamburg, Germany).

TABLE 1

| | | SEQ |
|---|---|---|
| | | ID |
| Name | Amino acid sequence | NO.: |
| mNeonGreen- | MGSSHHHHHHSSGLVPRGSHMMLPGCMLMV | 1 |
| HMG | SKGEEDNMASLPATHELHIFGSINGVDFDM | |
| | VGQGTGNPNDGYEELNLKSTKGDLQFSPWI | |
| | LVPHIGYGFHQYLPYPDGMSPFQAAMVDGS | |
| | GYQVHRTMQFEDGASLTVNYRYTYEGSHIK | |
| | GEAQVKGTGFPADGPVMTNSLTAADWCRSK | |
| | KTYPNDKTIISTFKWSYTTGNGKRYRSTAR | |
| | TTYTFAKPMAANYLKNQPMYVFRKTELKHS | |
| | KTELNFKEWQKAFTDVMGMDELYKLETPKR | |
| | PRGRPKKMLGSGC | |
| | (31.79 kDa) | |
| Truncated | MGSSHHHHHHSSGLVPRGSHMDLRTLGYSQ | 2 |
| TALE | QQQEKIKPKVRSTVAQHHEALVGHGFTHAH | |
| (tTALE)- | IVALSQHPAALGTVAVKYQDMIAALPEATH | |
| emGFP | EAIVGVGKQWSGARALEALLTVAGELRGPP | |
| | LQLDTGQLLKIAKRGGVTAVEAVHAWRNAL | |
| | TGAPLNLTPAQVVAIASNNGGKQALETVQR | |
| | LLPVLCQDHGLTPAQVVAIASNGGGKQALE | |
| | TVQRLLPVLCQAHGLTPDQVVAIASHDGGK | |
| | QALETVQRLLPVLCQDHGLTPAQVVAIASN | |
| | GGGKQALETVQRLLPVLCQAHGLTPDQVVA | |
| | IASNNGGKQALETVQRLLPVLCQAHGLTPA | |
| | QVVAIASNGGGKQALETVQRLLPVLCQDHG | |
| | GGSGGMVSKGEELFTGVVPILVELDGDVNG | |
| | HKFSVSGEGEGDATYGKLTLKFICTTGKLP | |
| | VPWPTLVTTFAYGLQCFARYPDHMKQHDFF | |
| | KSAMPEGYVQERTISFKDDGNYKTRAEVKF | |
| | EGDTLVNRIELKGTDFKEDGNILGHKLEYN | |
| | YNSHNVYITADKQKNGIKANFKIRHNIEDG | |
| | SVQLADHYQQNTPIGDGPVLLPDNHYLSTQ | |
| | SALSKDPNEKRDHMVLLEFVTAAGITLGMD | |
| | ELYK | |
| | (64.7 kDa) | |
| H-NS | MGSSHHHHHHSSGLVPRGSHMMSEALKILN | 3 |
| | NIRTLRAQARECTLETLEEMLEKLEVVVNE | |
| | RREEESAAAAEVEERTRKLQQYREMLIADG | |
| | IDPNELLNSLAAVKSGTKAKRAQRPAKYSY | |
| | VDENGETKTWTGQGRTPAVIKKAMDEQGKS | |
| | LDDFLIKQGSGC | |
| | (18.10 kDa) | |
| H-NS- | MGSSHHHHHHSSGLVPRGSHMMSEALKILN | 4 |
| mScarlet | NIRTLRAQARECTLETLEEMLEKLEVVVNE | |
| | RREEESAAAAEVEERTRKLQQYREMLIADG | |
| | IDPNELLNSLAAVKSGTKAKRAQRPAKYSY | |
| | VDENGETKWTGQGRTPAVIKKAMDEQGKSL | |
| | DDFLIKQEFMVSKGEAVIKEFMRFKVHMEG | |
| | SMNGHEFEIEGEGEGRPYEGTQTAKLKVTK | |
| | GGPLPFSWDILSPQFMYGSRAFTKHPADIP | |
| | DYYKQSFPEGFKWERVMNFEDGGAVTVTQD | |
| | TSLEDGTLIYKVKLRGTNFPPDGPVMQKKT | |
| | MGWEASTERLYPEDGVLKGDIKMALRLKDG | |
| | GRYLADFKTTYKAKKPVQMPGAYNVDRKLD | |
| | ITSHNEDYTVVEQYERSEGRHSTGGMDELY | |
| | KGSGC | |
| | (44.75 kDa) | |
| Cro- | MGSSHHHHHHSSGLVPRGSHMMEQRITLKD | 5 |
| mNeonGreen | YAMRFGQTKTAKDLGVYQSAINKAIHAGRK | |
| | IFLTINADGSVYAEEVKPFPSNKKTTAGGS | |
| | GGPGMVSKGEEDNMASLPATHELHIFGSIN | |
| | GVDFDMVGQGTGNPNDGYEELNLKSTKGDL | |
| | QFSPWILVPHIGYGFHQYLPYPDGMSPFQA | |
| | AMVDGSGYQVHRTMQFEDGASLTVNYRYTY | |
| | EGSHIKGEAQVKGTGFPADGPVMTNSLTAA | |
| | DWCRSKKTYPNDKTIISTFKWSYTTGNGKR | |
| | YRSTARTTYTFAKPMAANYLKNQPMYVFRK | |
| | TELKHSKTELNFKEWQKAFTDVMGMDELYK | |
| | GSGC | |
| | (37.07 kDa) | |

(DNA binding protein-polymer complex composition)

### Example 1: DNA-Truncated TALE (tTALE)-emGFP-PVP Complex Composition

A mixture was prepared by mixing 1 ng of λ phage DNA molecule (48.5 kb) with 396 nM Truncated TALE (tTALE)-emGFP, DNA binding protein-fluorescent protein according to Preparation Example 1 at room temperature for 10 minutes. The mixture was diluted with 1×TE buffer solution, and then an equal volume of a 5% aqueous solution of polyvinylpyrrolidine (PVP, 40 Kilodaltons) was added thereto and a reaction was allowed to occur therebetween for 5 minutes, thereby preparing a DNA-Truncated TALE (tTALE)-emGFP-PVP complex composition.

### Example 2: DNA-H—NS—PVP Complex Composition

A DNA-H—NS—PVP complex composition was prepared in the same manner as in Example 1, except that 264 nM H—NS according to Preparation Example 2 was used as a DNA-binding protein instead of 396 nM Truncated TALE (tTALE)-emGFP according to Preparation Example 1 as DNA binding protein-fluorescent protein.

### Example 3: DNA-H—NS-mScarlet-PVP Complex Composition

A DNA-H—NS-mScarlet-PVP complex composition was prepared in the same manner as in Example 1, except that 264 nM H—NS-mScarlet according to Preparation Example 3 was used as DNA binding protein-fluorescent protein instead of 396 nM Truncated TALE (tTALE)-emGFP according to Preparation Example 1.

### Example 4: DNA-Cro-mNeonGreen-PVP Complex Composition

A DNA-Cro-mNeonGreen-PVP complex composition was prepared in the same manner as in Example 1, except that 264 nM Cro-mNeonGreen according to Preparation

Example 4 was used as DNA binding protein-fluorescent protein instead of 396 nM Truncated TALE (tTALE)-emGFP according to Preparation Example 1.

### Example 5: DNA-mNeonGreen-HMG-PEOX Complex Composition

A DNA-mNeonGreen-HMG-PEOX complex composition was prepared in the same manner as in Example 1, except that 132 nM mNeonGreen-HMG according to Preparation Example 5 was used as DNA binding protein-fluorescent protein instead of 396 nM Truncated TALE (tTALE)-emGFP according to Preparation Example 1, and a 1% aqueous solution of poly 2-ethyl-2-oxazoline (PEOX, 50 Kilodaltons) was used instead of the 5% aqueous solution of PVP (40 Kilodaltons).

### Example 6: DNA-mNeonGreen-HMG-PANI Complex Composition

A DNA-mNeonGreen-HMG-PANI complex composition was prepared in the same manner as in Example 1, except that 396 nM mNeonGreen-HMG according to Preparation Example 5 was used as DNA binding protein-fluorescent protein instead of 396 nM Truncated TALE (tTALE)-emGFP according to Preparation Example 1, and a 0.1% aqueous solution of polyaniline (PANI) was used instead of the 5% aqueous solution of PVP (40 Kilodaltons).

### Example 7: DNA-mNeonGreen-HMG-PVP Complex Composition

A DNA-mNeonGreen-HMG-PVP complex composition was prepared in the same manner as in Example 1, except that 132 nM mNeonGreen-HMG according to Preparation Example 5 was used as DNA binding protein-fluorescent protein instead of 396 nM Truncated TALE (tTALE)-emGFP according to Preparation Example 1.

### Example 8: DNA-mNeonGreen-HMG-PVP Complex Composition

A DNA-mNeonGreen-HMG-PVP complex composition was prepared in the same manner as in Example 1, except that 1 ng of M13mp18 double-stranded DNA (7.2 kb) was used as a DNA molecule instead of 1 ng of λ phage DNA molecule (48.5 kb), and 132 nM mNeonGreen-HMG according to Preparation Example 5 was used as DNA binding protein-fluorescent protein instead of 396 nM Truncated TALE (tTALE)-emGFP according to Preparation Example 1.

### Comparative Example 1: DNA Solution

A DNA solution was prepared by diluting 1 ng of λ phage DNA molecule (48.5 kb) with 1×TE (Tris-EDTA) (Tris 10 mM EDTA 1 mM pH 8.0) buffer solution.

### Comparative Example 2: DNA-mNeonGreen-HMG Mixture

A DNA-mNeonGreen-HMG mixture was prepared by mixing 1 ng of λ phage DNA molecule (48.5 kb) with 792 nM mNeonGreen-HMG as DNA binding protein-fluorescent protein according to Preparation Example 5 at room temperature for 10 minutes.

### Comparative Example 3: DNA-PVP Mixture

A DNA-PVP mixture was prepared by adding 1 ng of λ phage DNA molecule (48.5 kb) to a 5% aqueous solution of PVP (40 Kilodaltons) and mixing the resulting solution at room temperature for 10 minutes.

### Comparative Example 4: DNA-PANI Mixture

A DNA-PANI mixture was prepared by adding 2.5 ng of λ phage DNA molecule (48.5 kb, 32 Megadaltons) to a 0.1% aqueous solution of polyaniline (PANI) and mixing the resulting solution at room temperature for 10 minutes.

### (Silicon Wafers Having Different Surfaces)

### Reference Example 1: Silicon Wafer Surface-Modified with Cations

A silicon oxide wafer with a 30 nm-thick silicon oxide film was purchased from wafer market (Yongin, Korea) and prepared. The silicon oxide wafer or glass coverslip was placed in a Teflon rack and immersed in a piranha etchant (30:70 v/v H₂O₂/H₂SO₄) for 3 hours. The wafer and the coverslip were thoroughly washed with deionized water, and then pH 7 paper was used to confirm whether the pH was neutral (pH 7). The wafer and the coverslip were then sonicated in deionized water for 30 minutes, and rinsed again in deionized water, to expose the piranha surface. To prepare a 1.1 mM solution, 150 μL of Q-siloxane (N-trimethoxysilylpropyl-N,N,N-trimethyl ammonium chloride) in 50% methanol was added to 250 ml of deionized water. The wafer and the coverslip were incubated at 65° C. and 100 rpm for 16 hours. Finally, the wafer was rinsed three times with 99.9% ethanol and stored in 99.9% ethanol to manufacture a silicon wafer surface-modified with cations.

### Reference Examples 2 to 4: Silicon Oxide Wafer, Silicon Wafer, and Pt-Coated Silicon Wafer

A silicon oxide wafer according to Reference Example 2, a silicon wafer according to Reference Example 3, and a Pt-coated silicon wafer according to Reference Example 4 were purchased from wafer market (Yongin, Korea) and prepared.

### Analysis Example 1: Observation of Stained DNA Molecules on Surfaces of Substrates Through FE-SEM

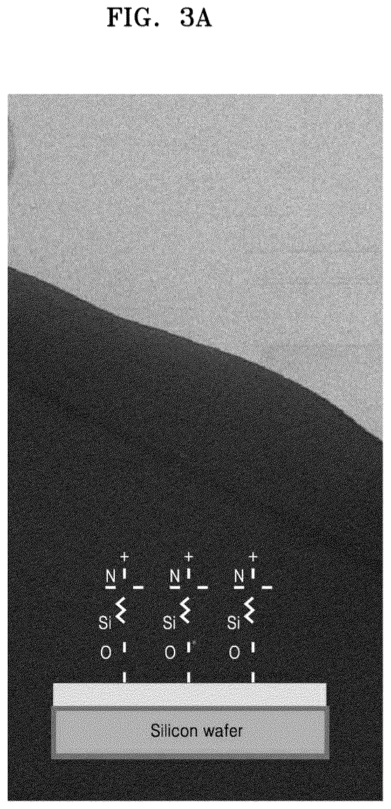

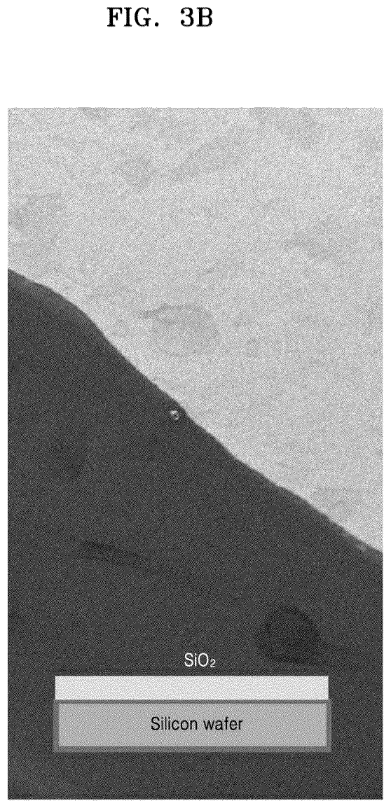

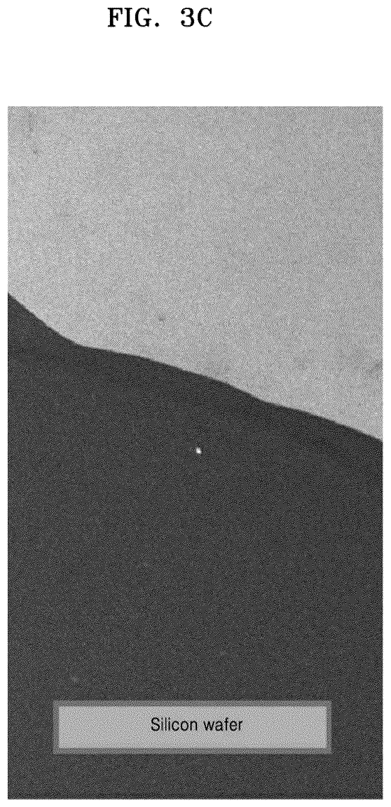

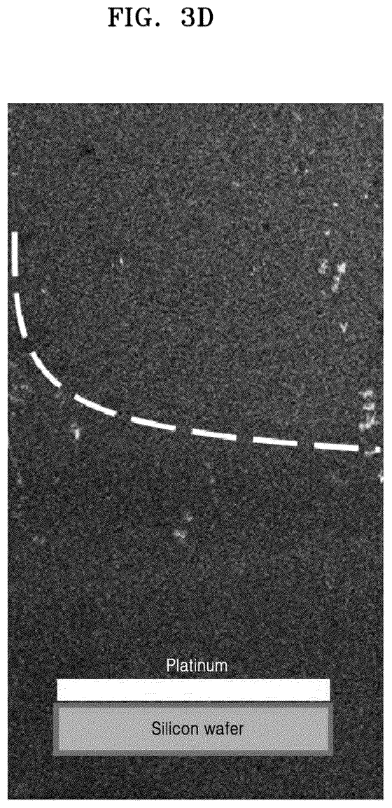

DNA molecules stained using the DNA-mNeonGreen-HMG-PVP complex composition according to Example 7 on surfaces of the silicon wafers according to Reference Examples 1 to 4 were observed through a field emission (FE)-scanning electron microscope (SEM) in the following manner. A microscope available from JSM-7100F, JEOL was used as the FE-SEM. The results thereof are illustrated in FIGS. 3A to 3D.

First, the complex composition was injected into an arrow portion of the PDMS microchannel device as described above (FIG. 2). Next, the DNA molecule of the complex composition was immobilized on and stretched from a surface of each of the silicon wafers according to Reference Examples 1 to 4. Next, before the PDMS microchannel device was detached from each of the silicon wafers according to Reference Examples 1 to 4, the complex composition inside a PDMS microchannel was dried for 50 minutes to observe the stained DNA molecule of the DNA-mNeonGreen-HMG-PVP complex.

Referring to FIGS. 3A to 3C, all boundaries between images (black) of the DNA-mNeonGreen-HMG-PVP on the surfaces of the silicon wafers according to Reference Examples 1-3 and images (gray) of adjacent silicon wafer areas were clearly distinguished. Among these, the image (black) of the DNA-mNeonGreen-HMG-PVP complex on the surface of the silicon wafer surface-modified with cations according to Reference Example 1 looked more clearly black, and the boundary between the image of the complex and the image (gray) of the adjacent silicon wafer area was also significantly and clearly distinguished.

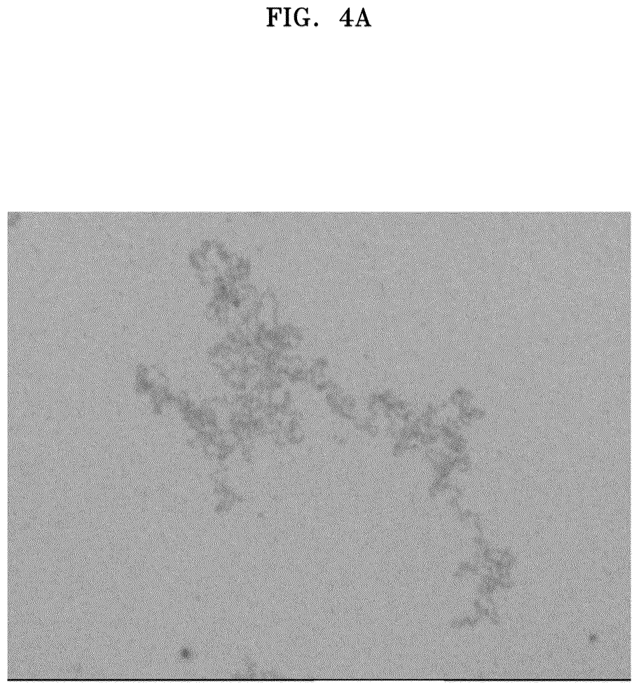

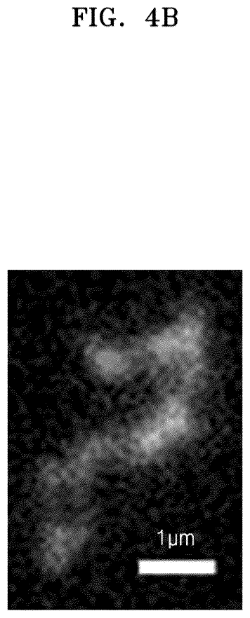

Referring to FIG. 4, both the image of the DNA-mNeonGreen-HMG-PVP complex on a surface of the Pt-coated silicon wafer according to Reference Example 4 and the image of an adjacent silicon wafer area looked black and thus it was difficult to distinguish the images from each other, and a boundary (a portion represented by a dotted line) between the images was also blurred and thus it was difficult to distinguish the images from each other.

The results obtained for the surfaces of the silicon wafers according to Reference Examples 1 to 3 are because the polymer PVP of the DNA-mNeonGreen-HMG-PVP complex appears in a dark form by interfering with the emission of secondary electrons from the surfaces of the silicon wafers, and as a result, the images of the DNA-mNeonGreen-HMG-PVP complex look black. The results obtained for the surface of the silicon wafer surface-modified with cations according to Reference Example 1 are because the polymer PVP of the DNA-mNeonGreen-HMG-PVP complex not only can interfere with the emission of secondary electrons from the surface of the silicon wafer surface-modified with cations, but can also rigidly immobilize DNA backbone, and as a result, the image (black) of the DNA-mNeonGreen-HMG-PVP complex looks clearer. From this, it can be confirmed that the silicon wafer surface-modified with cations serves to immobilize the DNA molecule of the DNA-protein-polymer complex.

In comparison, the results obtained for the surface of the Pt-coated silicon wafer according to Reference Example 4 are because, since the Pt-coated surface is fully conductive, the contrast between the complex and an adjacent silicon wafer area is reduced due to the surge and emission of secondary electrons capable of passing through a PVP-containing layer on the surface of the silicon wafer.

### Analysis Example 2: DNA Observation Depending on Whether PDMS Microchannel Device is Used

DNA molecules stained using the DNA-mNeonGreen-HMG-PVP complex composition according to Example 7 and the DNA-mNeonGreen-HMG-PEOX complex composition according to Example 5 were observed through an FE-SEM and a fluorescence microscope (FM)), without and with the PDMS microchannel device as follows. The results thereof are illustrated in FIGS. 4A, 4B, 5, 6A, and 6B.

Case without Using PDMS Microchannel Device

Each complex composition was applied onto a surface of the silicon wafer surface-modified with cations according to Reference Example 1 and dried, and then the stained DNA molecules of these complexes were observed through an FE-SEM and an FM.

Case with Using PDMS Microchannel Device

Each complex composition was injected into an arrow portion of the PDMS microchannel device (FIG. 2) described above, and then the DNA molecule of each complex was immobilized on and stretched from the surface of the silicon wafer surface-modified with cations according to Reference Example 1. Before the PDMS microchannel device was detached from the silicon wafer according to Reference Example 1, the complex composition inside a PDMS microchannel was dried for 50 minutes, and then the stained DNA molecule of the DNA-protein-polymer complex was observed through an FE-SEM.

### FE-SEM Equipment

An FE-SEM available from JEOL, JSM-7100F was used at 15 kV and a magnification of x 5000.

### FM Equipment

As an FM, an FM system consisting of an inverted microscope (Olympus IX70, Tokyo, Japan) equipped with a 100 × Olympus UplanSApo oil immersion objective and an LED light source for lighting (SOLA SM II Light Engine, Lumenko, Beaverton, OR) was used. Fluorescence images were captured by using a scientific-grade complementary metal-oxide-semiconductor digital camera (2048×2048, Prime sCMOS camera, Photometric, Tucson, AZ) and saved in 16-bit TIFF format by using Micro-manager software. ImageJ software was used for image processing, including overlay of multicolor images.

### 2-1. DNA-mNeonGreen-HMG-PVP Complex

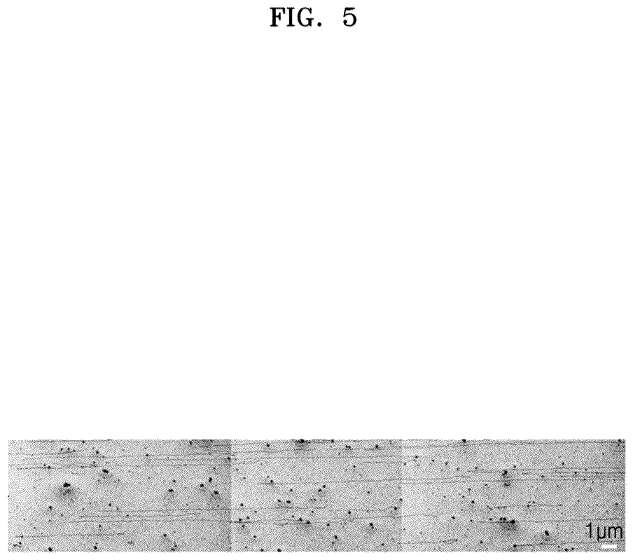

FIGS. 4A and 4B illustrate the results of observing DNA molecules stained using the DNA-mNeonGreen-HMG-PVP complex composition according to Example 7, through an FE-SEM and an FM, respectively, in which a PDMS microchannel device was not used. FIG. 5 illustrates the results of observing, through an FE-SEM, a DNA molecule stained using the DNA-mNeonGreen-HMG-PVP complex composition according to Example 7, in which a PDMS microchannel device was used.

Referring to FIGS. 4A and 4B, even though the PDMS microchannel device was not used, an image (FIG. 4A) of the stained DNA molecules of the DNA-mNeonGreen-HMG-PVP, observed through an FE-SEM, was clearly visible as a black line, compared with an image (FIG. 4B) of the stained DNA molecules of the DNA-mNeonGreen-HMG-PVP complex, observed through an FM.

Referring to FIG. 5, the stained DNA molecules of the DNA-mNeonGreen-HMG-PVP complex, observed through an FE-SEM with the PDMS microchannel device, were immobilized on the surface of the silicon wafer surface-modified with cations and appeared as parallel black lines.

### 2-2. DNA-mNeonGreen-HMG-PEOX Complex

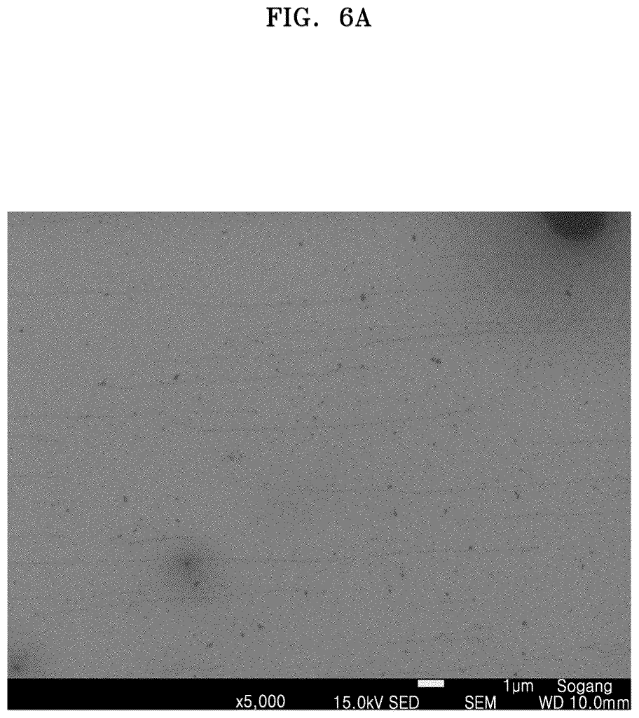

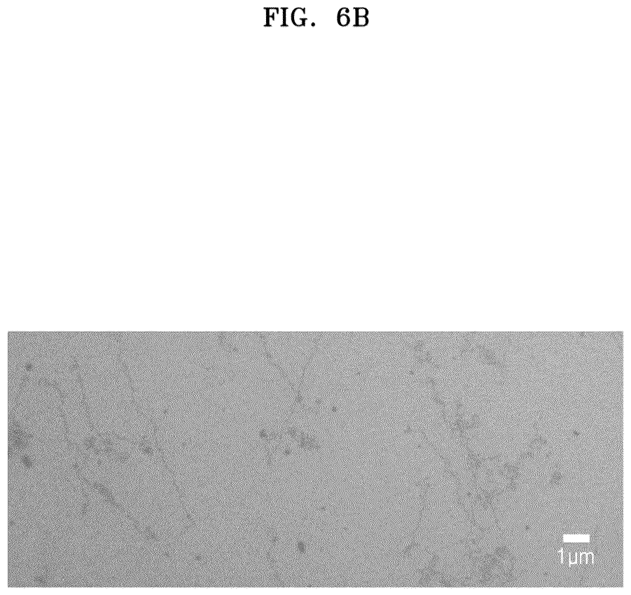

FIG. 6A illustrates the results of observing, through an FE-SEM, DNA molecules stained using the DNA-mNeonGreen-HMG-PEOX complex composition according to Example 5, in which a PDMS microchannel device was used. FIG. 6B illustrates the results of observing, through an FE-SEM, DNA molecules stained using the DNA-mNeonGreen-HMG-PEOX complex composition according to Example 5, in which a PDMS microchannel device was not used.

Referring to FIGS. 6A and 6B, DNA molecules stained using the DNA-mNeonGreen-HMG-PEOX complex composition according to Example 5, for which the PDMS microchannel device was used, were immobilized on the surface of the silicon wafer surface-modified with cations and appeared as parallel black lines. From this, it can be confirmed that the PDMS microchannel device serves to stretch the DNA molecules of the DNA-protein-polymer complex in parallel on the surface of the silicon wafer surface-modified with cations.

In comparison, the flow of DNA molecules stained using the DNA-mNeonGreen-HMG-PEOX complex composition according to Example 5 without using the PDMS microchannel device was not constant on the surface of the silicon wafer surface-modified with cations, and thus was not stretched in one direction, resulting in random arrangement.

### Analysis Example 3: DNA Observation by FE-SEM and/or FM for Complexes, Molecules, and Mixtures

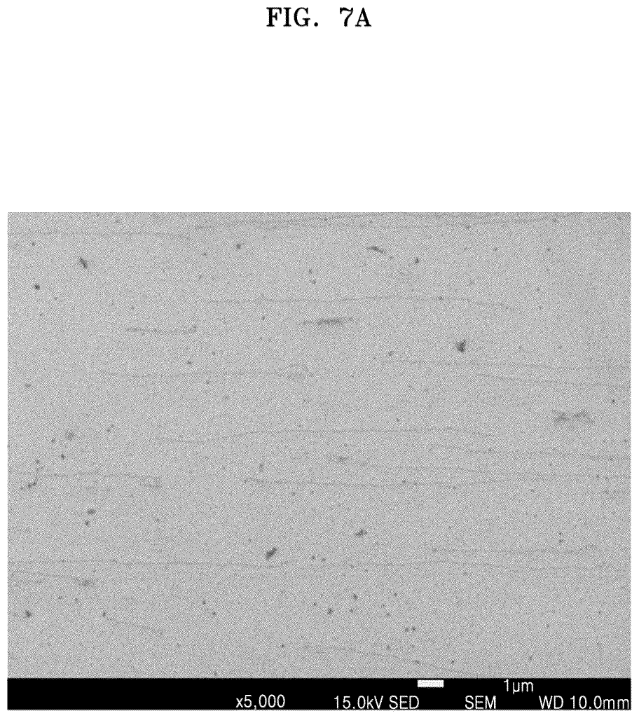

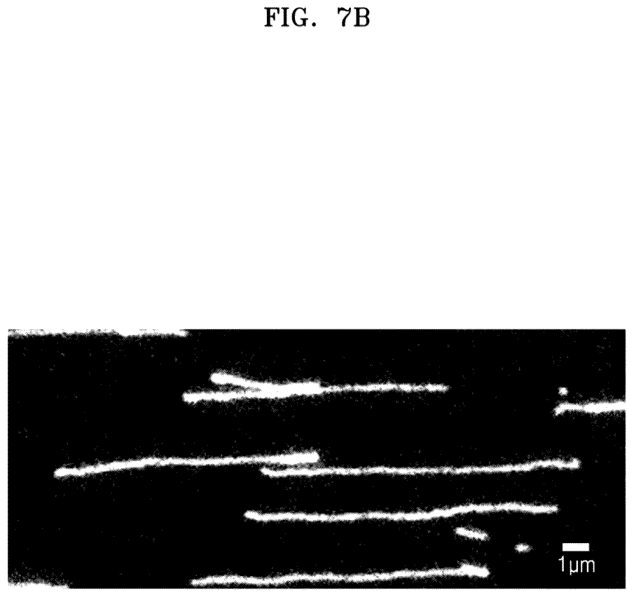

The DNA-protein-polymer complex compositions according to Examples 1 to 6, and the DNA molecules, DNA-protein mixture or DNA-polymer mixture according to Comparative Examples 1 to 4 were used, and stained DNA molecules on the silicon wafer surface-modified with cations according to Reference Example 1, with a PDMS microchannel device used, were observed through an FE-SEM and/or an FM. DNA observation was performed in the same manner as in the case where the PDMS microchannel device of Analysis Example 2 was used. The FE-SEM and the FM were the same as the equipment of Analysis Example 2. The results thereof are illustrated in FIGS. 7A, 7B, 8, 9A, 9B, 10A, 10B, 11, 12, 13A, 13B, 14, and 15.

### 3-1. DNA Observation for DNA-Protein-Polymer Complexes

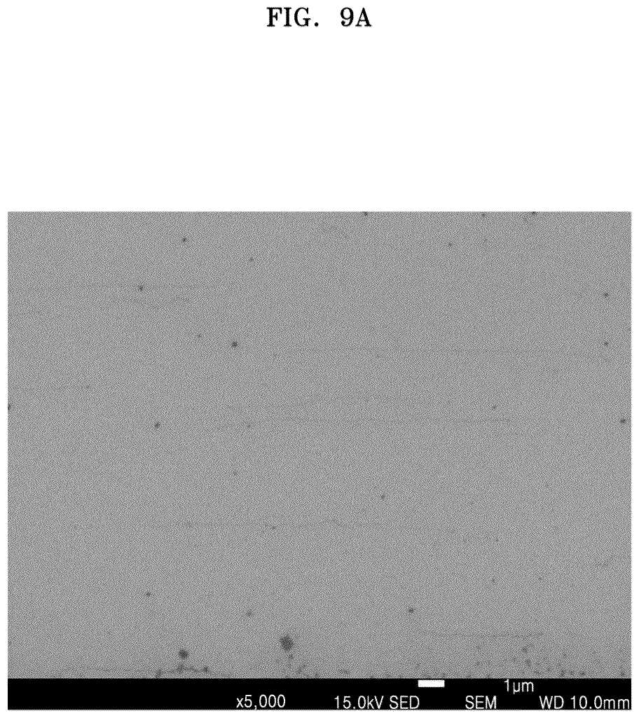

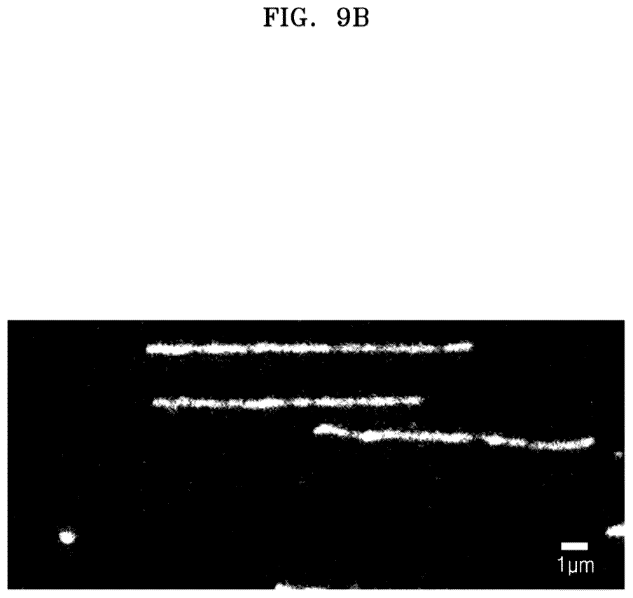

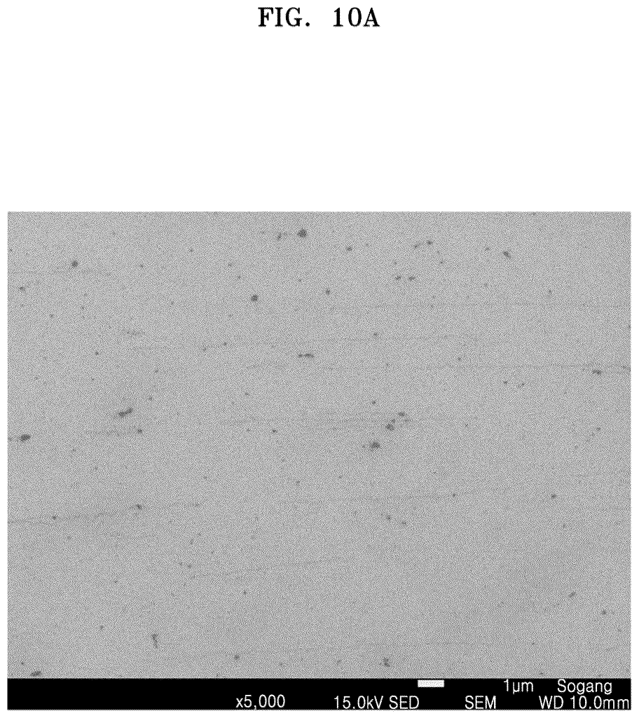

FIGS. 7A and 7B illustrate the results of observing DNA molecules stained using the DNA-protein-polymer complex composition according to Example 1, through an FE-SEM and an FM, respectively. FIGS. 9A and 9B illustrate the results of observing DNA molecules stained using the DNA-protein-polymer complex composition according to Example 3, through an FE-SEM and an FM, respectively. FIGS. 10A and 10B illustrate the results of observing DNA molecules stained using the DNA-protein-polymer complex composition according to Example 4, through an FE-SEM and an FM, respectively.

Referring to FIGS. 7A, 9A, and 10A, when DNA molecules stained using the DNA-protein-polymer complex compositions according to Examples 1, 3, and 5 were observed through an FE-SEM, the stained DNA molecules generally appeared as delicate, continuous black lines. In comparison, referring to FIGS. 7A, 9A, and 10A, when DNA molecules stained using the DNA-protein-polymer complex compositions according to Examples 1, 3, and 5 were observed through an FM, only some lines of the stained DNA molecules were visible and they appeared broken and thick.

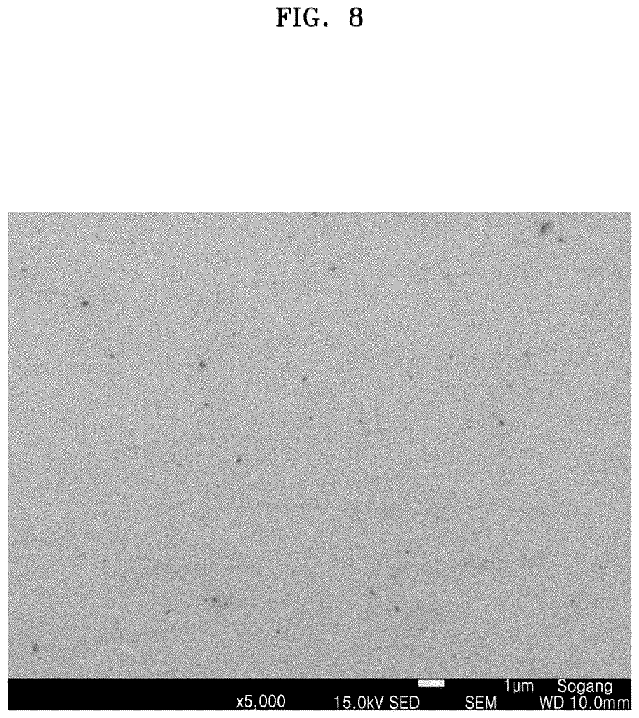

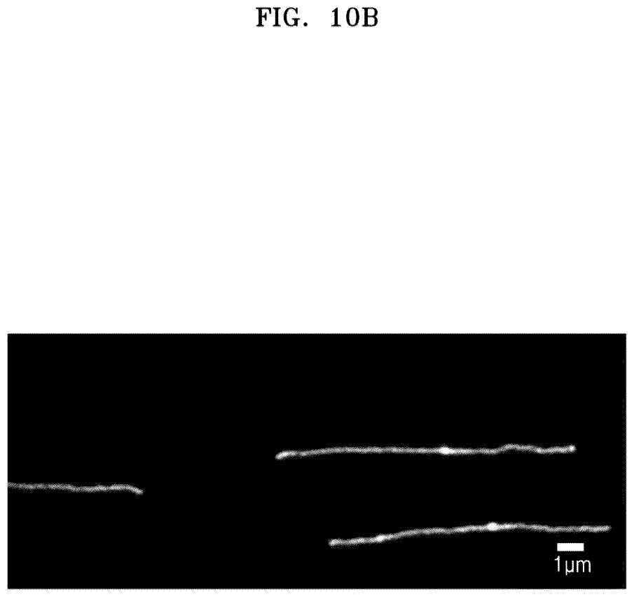

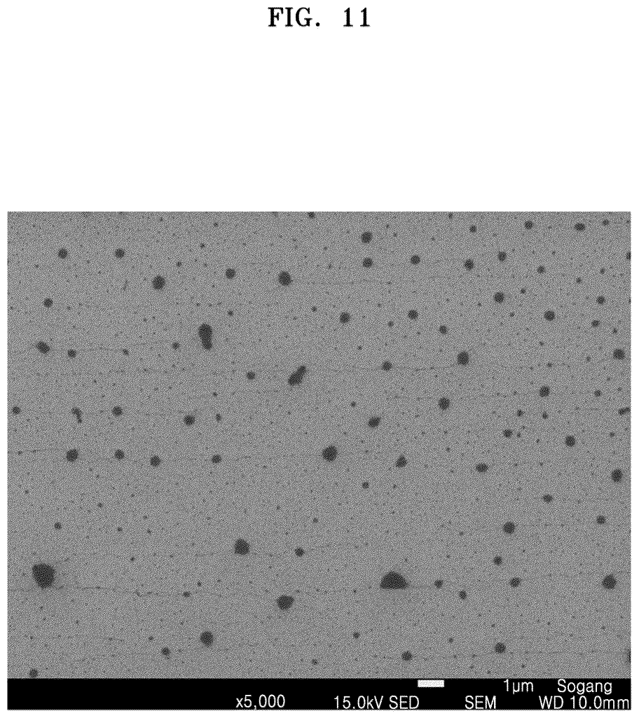

FIG. 8 illustrates the results of observing DNA molecules stained using the DNA-protein-polymer complex composition according to Example 2, through an FE-SEM. FIG. 11 illustrates the results of observing DNA molecules stained using the DNA-protein-polymer complex composition according to Example 6, through an FE-SEM.

Referring to FIGS. 8 and 11, when DNA molecules stained using the DNA-protein-polymer complex compositions according to Examples 2 and 6 were observed through an FE-SEM, the stained DNA molecules were slightly blurred but generally appeared as delicate, continuous black lines.

### 3-2. DNA Observation for DNA Molecules, DNA-Protein Mixture, and DNA-Polymer Mixture

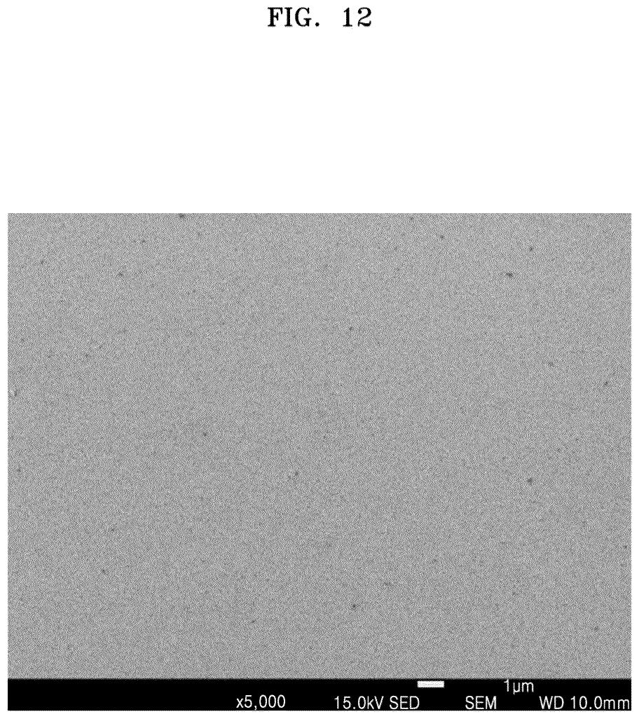

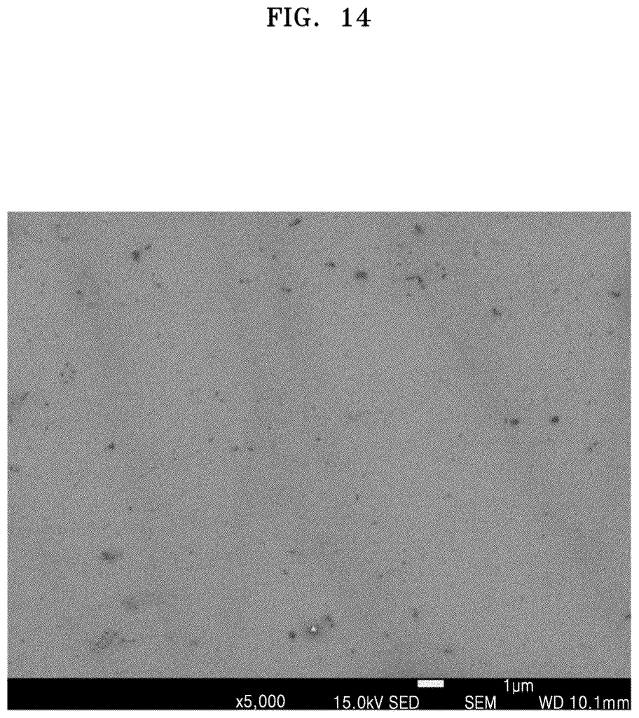

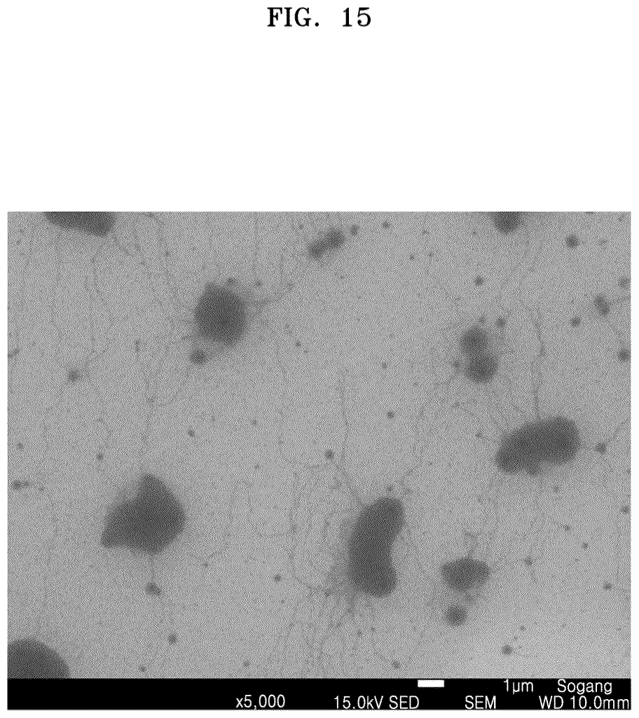

FIG. 12 illustrates the results of observing DNA molecules through an FE-SEM, in which the DNA solution according to Comparative Example 1 was used. FIG. 14 illustrates the results of observing stained DNA molecules through an FE-SEM, in which the DNA-PVP mixture according to Comparative Example 3 was used. FIG. 15 illustrates the results of observing stained DNA molecules through an FE-SEM, in which the DNA-PANI mixture according to Comparative Example 4 was used.

Referring to FIGS. 12, 14, and 15, when DNA molecules stained using the DNA solution and the DNA-polymer mixture were observed through an FE-SEM, the stained DNA molecules appeared as dots or randomly arranged solid lines.

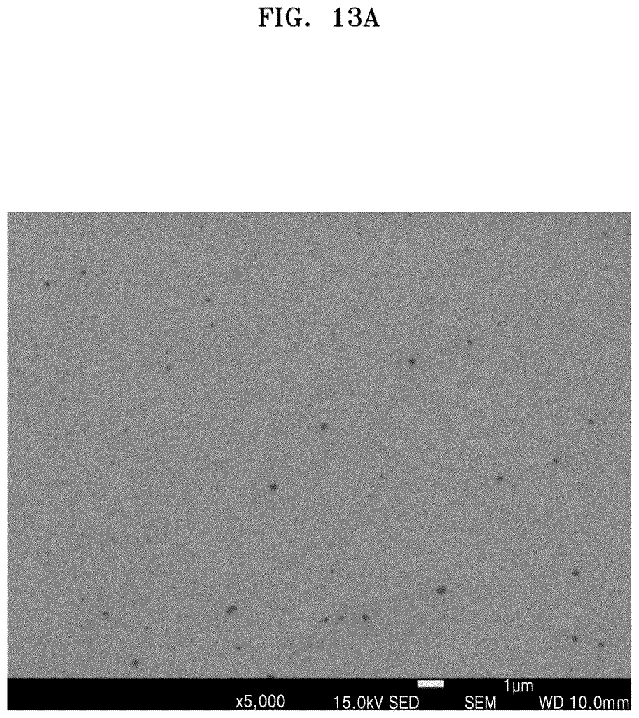

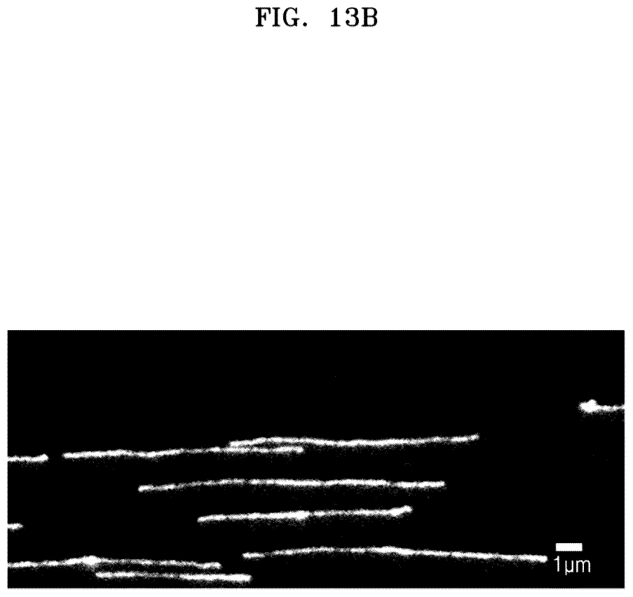

FIGS. 13A and 13B illustrate the results of observing DNA molecules through an FE-SEM and an FM, respectively, in which the DNA-mNeonGreen-HMG mixture according to Comparative Example 3 was used.

Referring to FIGS. 13A and 13B, when DNA molecules using the DNA-protein mixture were observed through an FE-SEM and an FM, the DNA molecules were not stained and appeared as dots or broken thick lines.

From this, it can be confirmed that images of well-arranged and stained DNA molecules can be acquired with high resolution through an FE-SEM, compared to an FM, and for effective electro-staining for DNA molecules, a DNA-binding protein capable of binding to the DNA molecules and a polymer component capable of binding to the DNA-binding protein are required.

### Analysis Example 4: Thickness and Full Width at Half Maximum (FWHM) of DNA-Protein-Polymer Complex by FE-SEM

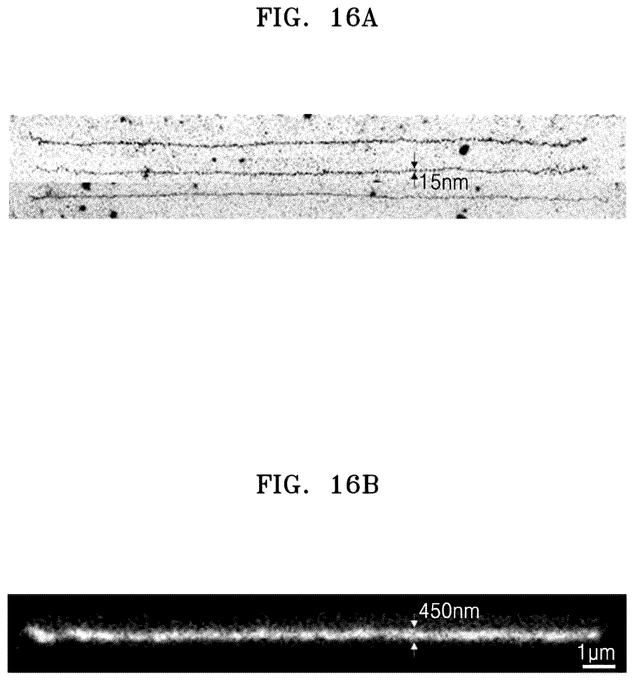

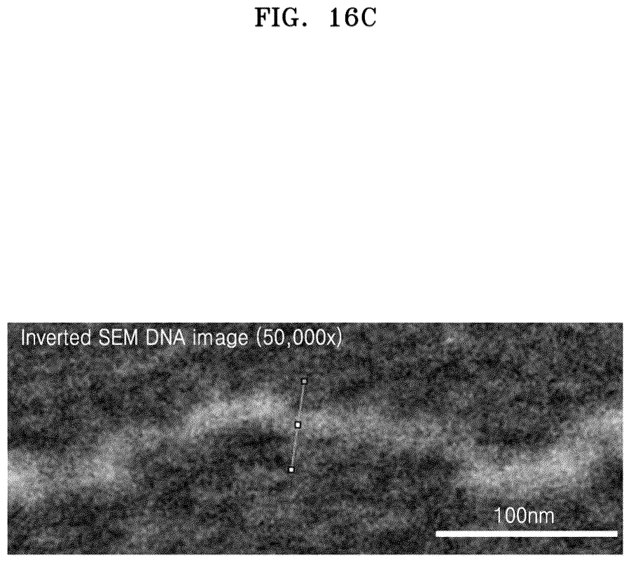

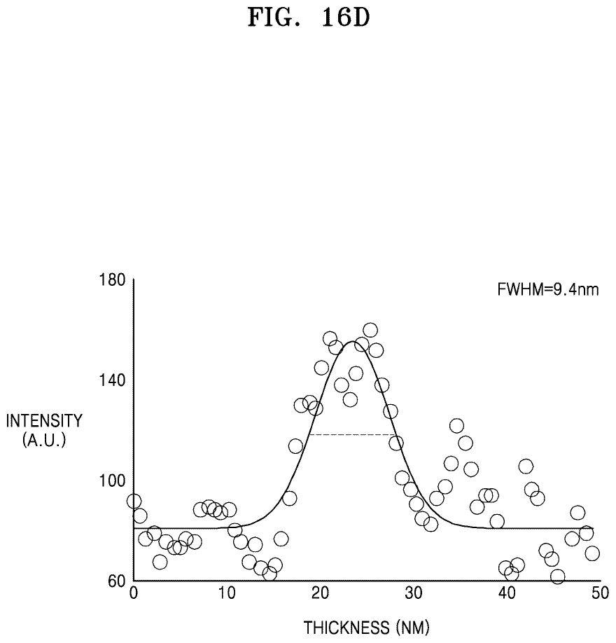

The DNA-mNeonGreen-HMG-PVP complex composition according to Example 7 was used and a PDMS microchannel device was used upon the silicon wafer surface-modified with cations according to Reference Example 1, to observe the thickness of a DNA-mNeonGreen-HMG-PVP complex through an FE-SEM and an FM. DNA observation was performed in the same manner as in the case where the PDMS microchannel device of Analysis Example 2 was used. The FE-SEM and the FM were the same as the equipment of Analysis Example 2. The results thereof are illustrated in FIGS. 16A and 16B.

Referring to FIGS. 16A and 16B, when DNA molecules stained using the DNA-mNeonGreen-HMG-PVP complex composition according to Example 7 were observed through an FE-SEM and an FM, respectively, high-resolution images of the stained DNA molecules were acquired as delicate, continuous black lines through an FE-SEM, compared to an FM, and the thicknesses of DNA-mNeonGreen-HMG-PVP were 15 nm and 450 nm, respectively, thus showing a big difference.

To accurately measure the thickness of the DNA-mNeonGreen-HMG-PVP complex, the full width at half maximum (FWHM) macro profile was used. To use the full width at half maximum (FWHM) macro profile, an inverted FE-SEM was used to measure the thickness of the DNA-mNeonGreen-HMG-PVP complex. The full width at half maximum (FWHM) macro was downloaded from GitHub to profile the intensity of DNA molecules observed as black lines through an FE-SEM, and the full width at half maximum (FWHM) of the profile was measured. The FWHM macro was used as an ImageJ plugin. The results thereof are illustrated in FIGS. 16C and 16D.

Referring to FIG. 16C, when the complex in the DNA-mNeonGreen-HMG-PVP complex composition according to Example 7 was observed through an inverted FE-SEM, the average FWHM (half width of a diagonal line) of the DNA-mNeonGreen-HMG-PVP complex was about 15.0±4.0 nm and in a range of 9.4 nm to 22.8 nm.

Referring to FIG. 16D, when the FWHM macro profile was used for the DNA-mNeonGreen-HMG-PVP complex according to Example 7, the FWHM of the complex was calculated to be 9.4 nm, which is the same as the results of observation through an inverted FE-SEM in FIG. 16C.

The FWHM was calculated on the basis of the following: DNA molecules having a thickness of 2 nm; an mNeonGreen-HMG protein having a diameter of 2.4 nm and a height of 4.2 nm; a PVP polymer having a weight average molecular weight (Mw) of 40 Kilodaltons; and R_{g} is 3.8 nm relative to a reference value in “Enhancement in Elastic Bending Rigidity of Polymer Loaded Reverse Microemulsion. Langmuir 2017, 33 (45): 13014-13026.” Assuming that the PVP polymer is circular, the outer diameter (dₒᵤₜ) of the PVP polymer was about 9.8 nm according to Equation 1 below. Aggregates of the protein and polymer surrounding the DNA molecules were shown to contribute greatly to the average FWHM (half width of a diagonal line) of the complex observed in FIG. 16C.

$${d}_{out} = 2 \sqrt{5 / 3} {R}_{g} \qquad Equation 1$$

From this, it can be confirmed that large DNA molecules having a thickness greater than typical DNA molecules can be observed through an FE-SEM, when stained using the DNA-protein-polymer complex.

### Analysis Example 5: Observation of Various DNA Shapes Through FE-SEM

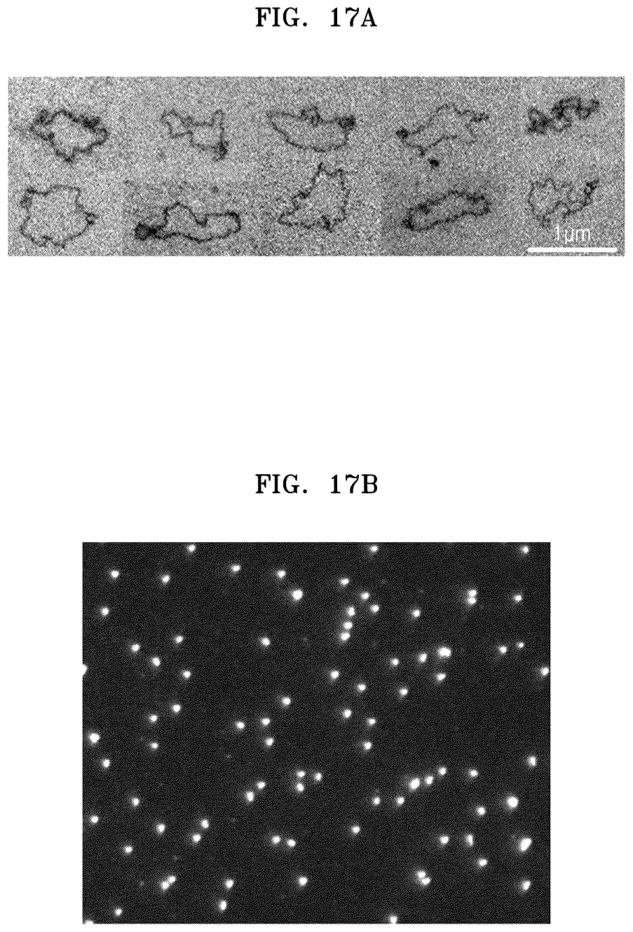

The DNA-mNeonGreen-HMG-PVP complex composition according to Example 8 was used and a PDMS microchannel device was used upon the silicon wafer surface-modified with cations according to Reference Example 1, to observe stained DNA molecules having various shapes through an FE-SEM and an FM. DNA observation was performed in the same manner as in the case where the PDMS microchannel device of Analysis Example 2 was used. The FE-SEM and the FM were the same as the equipment of Analysis Example 2. The results thereof are illustrated in FIGS. 17A and 17B.

Referring to FIG. 17A, when DNA molecules stained using the DNA-mNeonGreen-HMG-PVP complex composition was observed through an FE-SEM, the stained DNA molecules were shown to have a circular, helical, supercoil or double-stranded form, in addition to a linear form.

Referring to FIG. 17B, when DNA molecules stained using the DNA-mNeonGreen-HMG-PVP complex composition was observed through an FM, no clear shape could be seen and only dots were visible.

From this, it can be seen that DNA molecules having various shapes stained using the DNA-protein-polymer complex can be observed with high resolution through an FE-SEM.

Example embodiments have been described and illustrated in the accompanying drawings to facilitate the understanding of the disclosure. However, it should be understood that these embodiments are provided for illustrative purposes only and are not intended to limit the disclosure. It should also be understood that the disclosure is not limited to the illustrated and provided descriptions. This is because various other modifications can be made by those of ordinary skill in the art.

According to one or more embodiments, provided are a DNA analysis kit for analyzing DNA through scanning electron microscopy (SEM), a DNA-protein-polymer complex, a composition including the same, and a DNA analysis method using the DNA-protein-polymer complex.

The DNA-protein-polymer complex according to an embodiment includes: a DNA molecule; a DNA-binding protein including a peptide, the peptide having an amino acid sequence capable of binding to the DNA molecule and having at least one functional group at at least one of both terminuses thereof; and a polymer, an anhydride thereof or a salt thereof, capable of binding to the DNA-binding protein by intermolecular electrostatic attraction. The DNA-protein-polymer complex is metal-free and enables high-resolution observation and analysis of DNA molecules having various shapes and lengths at almost real-time speed through SEM.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A DNA analysis kit for analyzing DNA through scanning electron microscopy (SEM), the DNA analysis kit comprising:
a DNA-binding protein capable of binding to a DNA molecule through at least one of electrostatic interaction, intercalation, and groove binding; and
a polymer, an anhydride thereof or a salt thereof, capable of binding to the DNA-binding protein by intermolecular electrostatic attraction.

2. The DNA analysis kit of claim 1, wherein the DNA-binding protein comprises a fluorescent protein.

3. The DNA analysis kit of claim 1, wherein the polymer, the anhydride thereof, or the salt thereof comprises at least one of structural units represented by Formulae 1 to 9 and has average molecular weight of about 10 Kilodaltons to about 100 Kilodaltons. 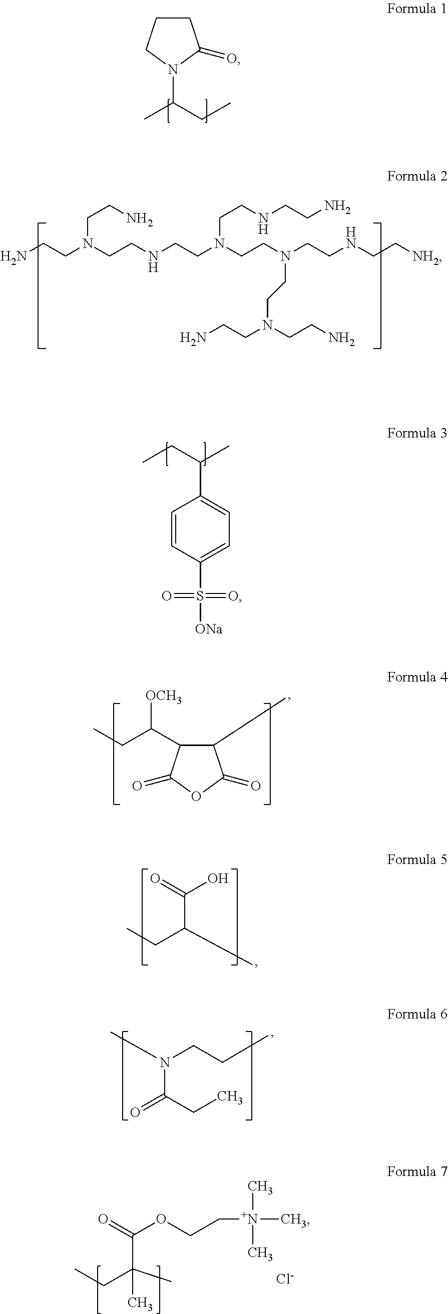 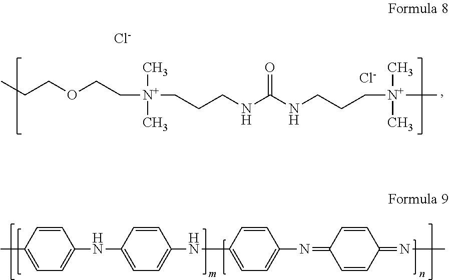
wherein, in Formulae 1 to 9,
m+n is 1.

4. A DNA-protein-polymer complex comprising:
a DNA molecule;
a DNA-binding protein comprising a peptide, the peptide having an amino acid sequence capable of binding to the DNA molecule and having at least one functional group in at least one of both terminuses thereof; and
a polymer, an anhydride thereof or a salt thereof, capable of binding to the DNA-binding protein by intermolecular electrostatic attraction.

5. The DNA-protein-polymer complex of claim 4, wherein the polymer comprises at least one of an oxygen atom and a nitrogen atom.

6. The DNA-protein-polymer complex of claim 5, wherein the oxygen atom or the nitrogen atom of the polymer is capable of binding to a hydrogen atom of an O—H bond or a hydrogen atom of an N—H bond, contained in the DNA-binding protein, by hydrogen bonding.

7. The DNA-protein-polymer complex of claim 4, wherein the polymer, the anhydride thereof, or the salt thereof is in the form of aggregates.

8. The DNA-protein-polymer complex of claim 4, wherein the polymer, the anhydride thereof, or the salt thereof comprises at least one of structural units represented by Formulae 1 to 9 and has average molecular weight of about 10 Kilodaltons to about 100 Kilodaltons: 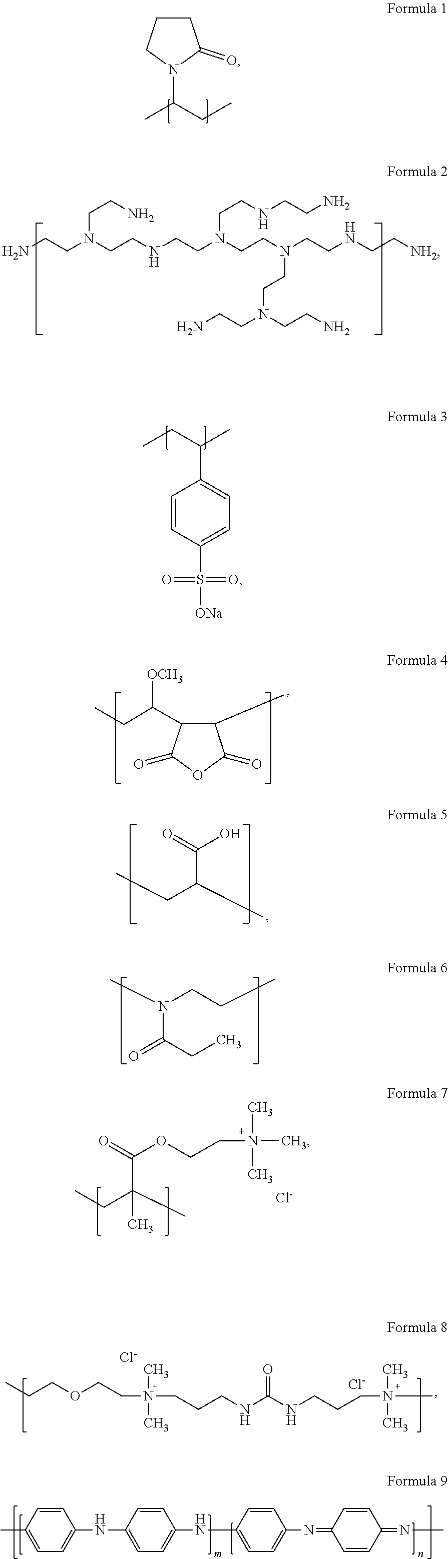
wherein, in Formulae 1 to 9,
m+n is 1.

9. The DNA-protein-polymer complex of claim 4, wherein the DNA molecule has a linear, circular, helical, supercoil, Christmas-tree, double-stranded or single-stranded form.

10. The DNA-protein-polymer complex of claim 4, wherein the DNA-binding protein comprises a fluorescent protein.

11. The DNA-protein-polymer complex of claim 4, wherein the DNA-binding protein is capable of binding to the DNA molecule through at least one of electrostatic interaction, intercalation, and groove binding.

12. The DNA-protein-polymer complex of claim 4, wherein the complex has a thickness of about 9 nm to about 20 nm.

13. The DNA-protein-polymer complex of claim 4, wherein the complex is used to stain DNA having a two-dimensional or three-dimensional form and enable visualization of the DNA through scanning electron microscopy (SEM).

14. The DNA-protein-polymer complex of claim 4, wherein the complex is used to enable visualization of DNA in a continuous form through scanning electron microscopy (SEM).

15. The DNA-protein-polymer complex of claim 4, wherein the complex is metal-free.

16. A composition comprising:
the DNA-protein-polymer complex according to claim 4; and
at least one selected from a solvent, an acid, a base, and a buffer solution.

17. A DNA analysis method using a DNA-protein-polymer complex, the method comprising:
providing a silicon substrate modified to have a positively charged surface; and
applying the composition according to claim 16 onto a surface of the silicon substrate and observing a DNA molecule through scanning electron microscopy.

18. The DNA analysis method of claim 17,
wherein the observing of the DNA molecule through scanning electron microscopy comprises:
immobilizing a DNA molecule on and stretching the DNA molecule from the surface of the silicon substrate by using a PDMS microchannel into which the composition is injected; and
removing the PDMS microchannel from the silicon substrate and observing the immobilized and stretched DNA molecule through scanning electron microscopy.

19. The DNA analysis method of claim 17,
wherein the providing of the silicon substrate comprises:
preparing a silicon substrate having a silicon oxide film formed thereon; and
contacting the silicon substrate having a silicon oxide film formed thereon with an ammonium salt precursor to thereby provide a silicon substrate modified to have a positively charged surface.

20. The DNA analysis method of claim 19, wherein the ammonium salt precursor comprises at least one selected from ammonia water, an ammonium chloride-based compound, an ammonium sulfate-based compound, an ammonium carbonate-based compound, an ammonium bicarbonate-based compound, and an ammonium acetate-based compound.
